(19) United States
(12) Patent Application Publication
(10) Pub. No.: US 2026/0074073 A1
(43) Pub. Date: Mar. 12, 2026

(54) Biomarker for Seronegative Rheumatoid Arthritis

(71) Applicant: MAYO FOUNDATION FOR MEDICAL EDUCATION AND RESEARCH, Rochester, MN (US)

(72) Inventors: Jaeyun Sung, Rochester, MN (US); John M. Davis, III, Rochester, MN (US); Benjamin Hur, Rochester, MN (US)

(21) Appl. No.: 19/320,153

(22) Filed: Sep. 5, 2025

Related U.S. Application Data
(60) Provisional application No. 63/691,705, filed on Sep. 6, 2024.

(51) Int. Cl. G16H 50/20; G16B 40/20

(52) U.S. Cl. CPC G16H 50/20; G16B 40/20

(57) ABSTRACT

The disclosure generally relates to methods of selecting a biomarker associated with a disorder or disease, and computer program products and systems for performing such methods. The disclosure further relates to biomarkers for rheumatoid arthritis and methods of use such biomarkers.

### CROSS-REFERENCE TO RELATED APPLICATIONS

This application claims the benefit under U.S.C. § 119(e) to U.S. Provisional Application No. 63/691,705 filed Sep. 6, 2024.

### FIELD

The present disclosure relates generally to the field of multi-omics data analysis, in particular, proteomics and metabolomics, machine learning and artificial intelligence to identify biomarkers for autoimmune disease diagnosis and treatment monitoring.

### BACKGROUND

Rheumatoid arthritis (RA) is a clinically important, chronic autoimmune inflammatory disease that is diagnosed in nearly 5 per 1000 adults worldwide (1-3). RA results in joint swelling, progressive joint destruction, pain, deformities, and potentially, bone erosion and cartilage destruction (4-6). Early diagnosis and treatment initiation of RA is crucial for improving patient outcomes in RA including preventing irreversible joint damage, disability, and the associated low quality of life. However, no specific set of signs and symptoms is pathognomonic for RA; instead, the diagnosis is made using a combination of clinical, laboratory, and imaging features.

In clinical practice, RA is diagnosed based on fulfilment of various classification criteria that have been defined by rheumatologic societies to identify discreet clinical features, inflammatory, and serological markers that can help guide diagnosis. For example, if a patient satisfied 4 out of 7 of clinical classification criteria set by the American College of Rheumatology (ACR) in 1987, they would be diagnosed with RA. This classification system includes scores for joints involvement, acute phase reactants (inflammatory markers like erythrocyte sedimentation rate (ESR) and C-reactive protein (CRP)), symptom duration and serology (for elevated autoantibodies). However, these criteria are limited by low sensitivity and poor specificity for targeting early arthritis, and therefore fail to identify the crucial population that would hopefully benefit from early therapeutic intervention.

The blood tests for elevated acute phase reactants such as the ESR and CRP indicate the general presence of an inflammatory state, but the tests are not specific to any disease process. Radiographic analysis of affected joints may also be performed. [Source—UptoDate and US20110052488A1] The autoantibody serological tests mainly evaluate the elevation of rheumatoid factors (RFs) and anti-citrullinated protein autoantibodies (ACPAs). For RA patients, the sensitivity of RF and ACPAs are similar (sensitivity ACPA IgG ˜67% and RF IgM ˜69%), however ACPAs are more specific for RA compared to RF (specificity of ACPA IgG ˜95% and RF IgM ˜85%) when compared to healthy controls (HC).

However, while RFs and ACPAs are often found in the serum of RA patients, not all RA patients have them. ACPA and RF are present in approximately 70%-80% of patients with RA; these patients are referred to as “seropositive”. However, an estimated 20%-25% of cases of RA do not present with RF and ACPA in serum despite meeting the clinical classification criteria for RA; these patients are referred to as “seronegative”. In sum, currently available laboratory tests for diagnosing RA have only moderate sensitivity and specificity and they have limited value for early diagnosis, subtype classification, and prognosis. Therefore, there is need for the identification of reliable diagnostic biomarkers for early diagnosis, subtype classification, and prognosis of RA.

Studies have for example suggested that ACPA- and ACPA-positive (ACPA+) RA are distinct disease subgroups that differ in their disease course and treatment response patterns (14, 15). Rapid advances in high-throughput molecular profiling approaches (“omics” technologies), have enabled the synchronous study of genes (genomics), RNA (transcriptomics), metabolites (metabolomics), proteins (proteomics), human-microbiota (microbiomics) on a global scale. These methodologies collectively advance our comprehension of RA pathways, and they are essential tools for the systemic characterization of biochemical entities e.g., protein, DNA, RNA, or metabolites, to provide a snapshot of the functional and pathophysiological states of an organism and support disease diagnosis and biomarker discovery.

However, the complexity of a biological system cannot be fully captured by a single-omics discipline. The integrated analysis of multiple single-omics modalities (multi-omics) from different layers of biological regulation has thus become a prevailing trend for constructing a comprehensive causal relationship between molecular signatures and phenotypic manifestations of a particular disease. Integrative approaches, by virtue of their ability to study the biological phenomenon holistically, can improve prognostics and predictive accuracy of disease phenotypes and hence can eventually aid in better treatment and prevention. Indeed, a multi-omic approach could enable the identification of intricate molecular signatures unique to ACPA− RA, which would have remained undetected in single-omic analyses. Furthermore, by capitalizing on the accessibility and minimally invasive nature of blood samples, multi-omic profiling in serum or plasma offers a highly promising opportunity for the development of novel diagnostic tools (23, 24).

The present disclosure addresses the shortfalls described above through a rigorous integrative multi-omic analysis approach to identify key biomarkers specific to ACPA− RA and ACPA+ RA. The disclosed methods demonstrate a promising strategy utilizing machine learning techniques to develop a next-generation molecular diagnostic blood test for seronegative RA.

### SUMMARY

The present disclosure provides methods, systems, combinations of tests, and collections of results useful for predicting whether an individual will develop Rheumatoid Arthritis (RA), the subtype of RA, and their treatment response. The disclosed methods, systems, combinations of tests, and collections of results are also useful for diagnosing the RA subtype of a subject suffering from RA, wherein the patient is afflicted with seropositive RA (ACPA+ RA) or wherein the patient is afflicted with seronegative RA (ACPA− RA), and further treating the subject based on their RA subtype.

Disclosed herein are methods for treating a subject afflicted with rheumatoid arthritis (RA), said method comprising the steps of diagnosing the subject with rheumatoid arthritis (RA), comprising: (1) obtaining a blood sample from the subject; and (b) determining the amount of a plurality of biomolecules comprising plasma proteins, plasma metabolites and plasma autoantibodies in the blood sample; wherein (i) the amount of at least 25 plasma proteins from Table A, and/or at least 25 plasma metabolites from Table B, and/or at least 25 plasma autoantibodies from Table C, or a combination of at least 50 biomolecules from Tables A, B and C, is higher in the blood sample relative to an amount of the biomolecules in a control sample from a subject without RA and indicates the subject has seronegative RA (ACPA− RA); or (ii) the amount of at least 25 plasma proteins from Table D, and/or at least 25 plasma metabolites from Table E, and/or at least 25 plasma autoantibodies from Table F, or a combination of at least 50 biomolecules from Tables D, E, and F, is higher in the blood sample relative to an amount of the biomolecules in a control sample from a subject without RA indicates the subject has seropositive RA (ACPA+ RA); and (c) administering at least one therapy to the subject.

In various embodiments, disclosed herein is a method of treating a subject afflicted with seronegative RA (ACPA− RA), said method comprising the steps of diagnosing the subject with seronegative RA (ACPA− RA), comprising: (a) obtaining a blood sample from the subject; and (b) determining the amount of biomolecules comprising plasma proteins, plasma metabolites and plasma autoantibodies in the blood sample; (i) wherein the amount of at least 25 biomolecules from Tables A, B, and C, is higher in the blood sample relative to an amount of the biomolecules in a control sample from a subject without RA and indicates the subject has seronegative RA (ACPA− RA); and (c) administering at least one therapy to the subject.

In still further embodiments, disclosed herein is a method of treating a subject afflicted with seropositive RA (ACP+ RA), said method comprising the steps of diagnosing the subject with seropositive RA (ACPA+ RA), comprising: (a) obtaining a blood sample from the subject; (b) determining the amount of biomolecules comprising plasma proteins, plasma metabolites and plasma autoantibodies in the blood sample; wherein the amount of at least 25 biomolecules from Tables D, E, and F, is higher in the blood sample relative to an amount of the biomolecules in a control sample from a subject without RA indicates the subject has seropositive RA (ACPA+ RA); and (c) administering at least one therapy to the subject.

In some embodiments, the determining the amount of biomolecules in step (b) of the aforementioned methods produces a multi-omic data set, wherein the multi-omic data set is processed through a trained machine learning model, and wherein the processed multi-omic data set provides a RA phenotype classification.

In some embodiments, disclosed herein is a method of diagnosing a subject with rheumatoid arthritis (RA), said method comprising the steps of preparing a RA phenotype classification network comprising: (a) receiving multi-omic data from a blood sample from a subject, wherein the multi-omic data includes proteomic, metabolomic, and autoantibody profiles; (b) processing the received multi-omic data through a trained machine learning model; (c) classifying the subject's RA phenotype based on the processed multi-omic data; and (d) outputting the classification result; and wherein: (i) the classification is ACPA− RA when the amount of at least 25 plasma proteins from Table A, and/or at least 25 plasma metabolites from Table B, and/or at least 25 plasma autoantibodies from Table C, or a combination of at least 50 biomolecules from Tables A, B, and C is detected; or (ii) the classification is ACPA+ RA when the amount of at least 25 plasma proteins from Table D, and/or at least 25 plasma metabolites from Table E, and/or at least 25 plasma autoantibodies from table F, or a combination of at least 50 biomolecules from Tables D, E, and F is detected. In various embodiments, the trained machine learning model is trained by: (a) selecting a cohort of subjects comprising subgroups of ACPA− RA patients, ACPA+ RA patients, and control individuals without RA; (b) collecting blood samples from the selected cohort; (c) performing deep multi-omic profiling on the collected blood samples, wherein the deep multi-omic profiling includes proteomic profiling, metabolomic profiling, and autoantibody profiling; (d) analyzing the multi-omic data to identify biomolecular features associated with each RA subgroup through statistical analyses, set comparisons, and network inference techniques; (e) constructing a global multi-omic network based on the identified biomolecular features and their associations with clinical attributes, including RA phenotypes; (f) applying a network diffusion technique to prioritize features closely linked to the RA phenotypes; thereby training the machine learning model using the prioritized features to distinguish between ACPA− RA subjects, ACPA+ RA subjects, and control subjects.

In still further embodiments, disclosed herein is a method of treating a subject with rheumatoid arthritis (RA), said method comprising the steps of: (a) diagnosing a subject with rheumatoid arthritis (RA), said method comprising the steps of preparing a RA phenotype classification network comprising: (a) receiving multi-omic data from a blood sample from a subject, wherein the multi-omic data includes proteomic, metabolomic, and autoantibody profiles; (b) processing the received multi-omic data through a trained machine learning model; (c) classifying the subject's RA phenotype based on the processed multi-omic data; and (d) outputting the classification result; and wherein: (i) the classification is ACPA− RA when the amount of at least 25 plasma proteins from Table A, and/or at least 25 plasma metabolites from Table B, and/or at least 25 plasma autoantibodies from Table C, or a combination of at least 50 biomolecules from Tables A, B, and C is detected; or (ii) the classification is ACPA+ RA when the amount of at least 25 plasma proteins from Table D, and/or at least 25 plasma metabolites from Table E, and/or at least 25 plasma autoantibodies from Table F, or a combination of at least 50 biomolecules from Tables D, E, and F is detected; and (e) administering at least one therapy to the subject.

In some embodiments, the subject is a mammal. In various embodiments, the mammal is a human.

In some embodiments, the determining the amount of plasma proteins comprises an aptamer-based assay technique. In various embodiments, the determining the amount of plasma proteins comprises an aptamer-based assay that measures binding of target proteins to aptamers in relative fluorescent units (RFUs). In some embodiments, the determining the amount of plasma metabolites comprises a mass spectrometry technique. In various embodiments the determining the amount of plasma proteins comprises ultra-high-performance liquid chromatography-tandem mass spectrometry (UPLC-MS/MS). In some embodiments, the determining the amount of plasma autoantibodies comprises a microarray-based assay technique.

In some embodiments, the therapy comprises administering one or more of a small molecule agent, immunotherapy, disease-modifying antirheumatic drugs (DMARDs), nonsteroidal anti-inflammatory drugs (NSAIDs), steroids, a biologic agent, and surgery. In various embodiments, the therapy comprises ibuprofen and/or naproxen sodium. In still further embodiments, the therapy comprises a corticosteroid comprising one or more of cortisone, prednisolone, methylprednisolone, dexamethasone, betamethasone, and hydrocortisone. In some embodiments, the therapy comprises at least one DMARD comprising methotrexate, leflunomide, hydroxychloroquine, and sulfasalazine. In various embodiments, the therapy comprises at least one of abatacept, adalimumab, anakinra, certolizumab, etanercept, golimumab, infliximab, rituximab, sarilumab, tocilizumab, baricitinib, tofacitinib, and Upadacitinib. In still further embodiments, the therapy comprises snynovectomy, tendon repair, joint fusion, and joint replacement. In some embodiments, 1, 2, 3, 4 or more therapies are administered.

### BRIEF DESCRIPTION OF THE DRAWINGS

FIGS. 1A-1C are a schematic overview of the multi-omic data integration and analytical framework provided by the devices, compositions, kits, and methods described herein. FIG. 1A shows the study design where deep multi-omic profiling of plasma samples collected from 40 ACPA− RA patients, 40 ACPA+ RA patients, and 40 healthy controls was used to create a dataset comprising 9,944 molecular features (proteins, metabolites, and autoantibodies). FIG. 1B shows the data analysis workflow including the set comparisons that were performed to characterize and differentiate the three study cohorts. A multi-omic network that identifies associations between various omic features and clinical attributes, including study group (or phenotype), was constructed using penalized (elastic net) linear regression. FIG. 1C shows the computational strategy for phenotype classification between study groups using a three-step feature selection scheme as follows: i) Multi-omic network construction: Nodes represent omic features, and edges symbolize links between features inferred using elastic net linear regression. This step forms the backbone of the network, laying the groundwork for subsequent steps. ii) Network topology propagation via a random walker algorithm: Initiated from a seed node representing the clinical phenotype (i.e., ACPA− RA, ACPA+ RA, or controls), this process uses a random walker algorithm (specifically a random walk with restart) to refine the initial full network into a smaller subnetwork composed of features most closely connected to the seed node. iii) Evaluation in 5-fold cross-validation: A random forest classifier was trained on the identified omic features to evaluate their potential for phenotype classification.

FIGS. 2A-2D provide plots showing the correlation between the multi-omic profiles and clinical characteristics is significantly different between ACPA− RA and ACPA+ RA. FIG. 2A provides ternary plots depicting feature abundances (mean values) in ACPA− RA, ACPA+ RA, and controls. Each point in the simplex triangle corresponds to a particular feature. The coordinates represent proportions (i.e., percentages) of mean abundances of each feature. Proteins display the greatest variation, while autoantibody abundances are the most consistent (or least variable) across the study groups. FIGS. 2B-2D provides rank plots of Spearman's rank correlation coefficient (?) between the molecular features and clinical characteristics (ESR, CRP, and DAS28-CRP). Features are sorted in ranked descending order of ? values (from positive to negative) in ACPA− RA. Each plot selectively visualizes the top ˜50 positive and negative correlations to illustrate the extremes of the distribution. The clear separation of gold points (ACPA− RA) from gray points (ACPA+ RA) in each tick on the x-axis (which correspond to the same feature for both gold and gray points) signifies substantial differences in both the most positive and negative correlations. ESR, erythrocyte sedimentation rate; CRP, C-reactive protein; DAS28-CRP, Disease Activity Score 28 using C-reactive protein.

FIGS. 3A-3D. Identification of differentially abundant proteins in ACPA− RA and ACPA+ RA plasma samples. FIGS. 3A-3C provides volcano plots showing the pairwise comparison of the differential abundant proteins in ACPA− RA vs. controls, ACPA+ RA vs. controls, ACPA+ RA vs. ACPA− RA samples. Among the 7,273 measured proteins, those that pass a P-value threshold (i.e., corresponding regression coefficient of P<0.05 in a model adjusted for potential confounders) and an effect size threshold (i.e., |Cohen's d|>0.5), were considered to be significantly associated with the corresponding phenotype or differentially abundant between study groups. Age, sex, BMI, smoking history, use of prednisone, bDMARDs, and csDMARDs were considered to be potential confounders. FIG. 3D is a Venn diagram illustrating the overlap and uniqueness of the differentially abundant proteins identified in FIG. 3A. FIG. 3E shows box and whiskers plots of the eight cytokines identified to be associated with at least one study group in one or more group comparisons. Each point in the boxplots represents a cytokine measured in normalized Relative Fluorescence Units (RFUs) in each plasma sample. An asterisk (*) denotes a significant difference (P<0.05 for the regression coefficient and |Cohen's d|>0.5) in protein abundance between comparison groups. FIG. 3F-3G provides bar graphs of Gene Ontology (GO) enrichment analysis of biological processes for up- and down-regulated proteins in ACPA− RA or ACPA+ RA compared to controls. While common immune-related enrichments were observed, both RA subgroups exhibit an over-representation of different metabolic processes. GO terms with P<0.05 (determined by a modified one-tailed Fisher's exact test) were considered as significantly enriched.

FIGS. 4A-4F. Plasma metabolomic analysis reveals ACPA status-/age-dependent differences in RA. FIG. 4A-4C provides volcano plots showing the differentially abundant metabolites associated with ACPA− RA, ACPA+ RA, and controls. Among the 1,061 metabolites analyzed, those demonstrating a significant regression coefficient (P<0.05 in a model adjusted for potential confounders) and an effect size of |Cohen's d|>0.5 were considered to be significantly associated with the corresponding phenotype, i.e., differentially abundant between study groups. FIG. 4D is a Venn diagram showing metabolites associated with the three study groups. FIG. 4E is an annotated bubble diagram of a KEGG subpathway enrichment analysis for the differentially-accumulated metabolites associated with ACPA− and ACPA+ RA identified in FIG. 4A. Bubble size denotes the number of differentially abundant metabolites annotated to that subpathway, and bubble color denotes the P-value of the subpathway enrichment. Only subpathways with P<0.05 (by a modified one-tailed Fisher's exact test) and with at least 2 significantly associated metabolites were considered. FIG. 4F provides boxplot-visualizations for a subset of 19 complex lipids, categorized into five subclasses of phospholipids and sphingomyelin, that showed higher abundance in the oldest age group of ACPA− RA (n=14) compared to the same age group of ACPA+ RA (n=14). An asterisk (*) indicates significant differences in lipids between the two RA subgroups. PI1, 1-palmitoyl-2-arachidonoyl-GPI (16:0/20:4); PI2, 1-stearoyl-2-arachidonoyl-GPI (18:0/20:4); PI3, 1-palmitoyl-2-oleoyl-GPI (16:0/18:1); PE1, 1-palmitoyl-2-arachidonoyl-GPE (16:0/20:4); PE2, 1-palmitoyl-2-oleoyl-GPE (16:0/18:1); PE3, 1-palmitoyl-2-stearoyl-GPE (16:0/18:0); PE4, 1-stearoyl-2-arachidonoyl-GPE (18:0/20:4); PE5, 1-oleoyl-2-arachidonoyl-GPE (18:1/20:4); PC1, 1-palmitoyl-2-oleoyl-GPC (16:0/18:1); PC2, 1-palmitoyl-2-palmitoleoyl-GPC (16:0/16:1); PC3, 1-stearoyl-2-oleoyl-GPC (18:0/18:1); PC4, 1,2-dipalmitoyl-GPC (16:0/16:0); LP1, 1-stearoyl-GPE (18:0); LP2, 1-palmitoyl-GPG (16:0); LP3, 2-palmitoyl-GPC (16:0); LP4, 2-stearoyl-GPE (18:0); LP5, 1-palmitoleoyl-GPC (16:1); LP6, 1-arachidonoyl-GPE (20:4n6); SM1, sphingomyelin (d18:2/16:0, d18:1/16:1).

FIGS. 5A-5D show that ACPA− RA and ACPA+ RA plasma samples have distinct autoantibody profiles. FIGS. 5A-5C provides volcano plots of autoantibodies associated with study groups identified through three pair-wise comparative analyses (i.e., ACPA− RA vs. controls, ACPA+ RA vs. controls, ACPA+ RA vs. ACPA− RA). Among 1,610 autoantibodies, those with an adjusted regression model coefficient of P<0.05 and an effect size of |Cohen's d|>0.2 were considered to be differentially abundant. FIG. 5D are heatmaps showing the Spearman's rank correlation coefficients (?) of plasma autoantibody abundances and clinical characteristics (ACPA titer, RF titer, DAS28-CRP, ESR, CRP) in ACPA− and ACPA+ RA. Only autoantibodies demonstrating a significant correlation (i.e., |?|>0.4 and P<0.01) with at least one parameter are represented in the heatmap, as indicated by an asterisk (*).

FIGS. 6A-6B are a multi-omics integration network analysis of the key predictors of the RA subgroup. FIG. 6A is an integration network visualizing predictive associations between omic features and phenotype (or study group), identified using elastic net penalized regression. Associations are represented by edges (gray lines), where non-zero regression coefficients signify a link between variables. All edges in the network have equal weight. Omic features closely linked to the phenotype (blue node located at the top) were calculated using a random walk with restart (RWR) approach. Specifically, the phenotype was used as the starting seed for RWR, with a 50% chance of being the restart location of the random walker (restart probability r=0.5). For visual clarity, a focused subnetwork comprising the top 30 variables with the closest associations to the phenotype node is shown. FIG. 6B is a heatmap detailing the mean Z-scores for a feature within a specific phenotype. Z-scores were calculated from the distribution across all samples (n=120). Asterisks (*) represent features that were previously found to be differentially abundant in the corresponding RA subgroup compared to controls.

FIG. 7 is a schematic showing the validation process for the ACPA− RA vs. ACPA+ RA classification model using independent plasma metabolomic datasets. The model development started with a training dataset comprising 80 plasma metabolomic samples (40 from ACPA− and 40 from ACPA+ RA patients). Network inference and network diffusion-based feature selection methods were used to optimize a subnetwork size and a random seed through 5-fold cross-validation. Only metabolites found in the training set were retained for analysis in each of the two independent validation datasets. A random forest classifier, configured with the best-performing subnetwork size and seed determined during internal validation (i.e., cross-validation on the training set), was then tested on these independent datasets. To address class imbalance, each validation set used random sampling (100 iterations) for performance testing; for example, in Independent dataset #1, all 9 ACPA− samples and a randomly chosen subset of 9 ACPA+ RA samples were used for each iteration. The classifier's performance, averaged over the iterations, was reported for each dataset. ENET, elastic net; RWR, random walk with restart; RF, random forest. [Highlighted text should be in the Examples, will cross check for text duplication]

### DETAILED DESCRIPTION

A hallmark diagnostic indicator for rheumatoid arthritis (RA) is the presence of anti-citrullinated protein antibodies (ACPA) in blood, yet approximately 40% of patients with RA are seronegative, i.e., test negative for ACPA (ACPA− RA). Recent evidence suggests that patients with ACPA− RA exhibit disease courses differently from patients with ACPA+ RA. However, the biomolecular distinctions of these two RA subgroups remain understudied, particularly in peripheral blood. The multi-omics approach disclosed herein elucidates the distinctions between ACPA− and ACPA+ RA by analyzing plasma proteomics, metabolomics, and autoantibody profiles from 120 individuals (40 ACPA− RA, 40 ACPA+ RA, and 40 healthy controls). While the statistical analyses identified several circulating biomolecules associated with both RA subgroups compared to controls, key differences (between subgroups) in cytokines, complement proteins, metabolites, and enriched biological processes were also observed. Additionally, opposite omic feature correlation patterns between ACPA− and ACPA+ RA were found, revealing further evidence of their phenotypic differences. Furthermore, the disclosed machine learning strategy, which utilizes an integrative network inference method and network diffusion-based feature selection scheme, was able to effectively discriminate between RA subgroups and controls based on plasma multi-omic profiles: classification AUCs of 0.95 for ACPA− RA vs. controls, 0.93 for ACPA+ RA vs. controls, and 0.91 for both RA subgroups vs. controls in 5-fold cross-validation. For differentiating ACPA− from ACPA+ RA, plasma metabolomics reached AUCs of 0.70-0.72 in both cross-validation and two independent validation datasets. The disclosure represents the most comprehensive analysis to date of blood proteomics, metabolomics, and autoantibody profiles in ACPA− and ACPA+ RA patients, offering a holistic view of the biomolecular variations between these subgroups that share the same clinical diagnosis. The results illuminate unique aspects of the understudied ACPA− RA phenotype, laying the groundwork for machine learning-based, molecular diagnostic blood tests to aid in the diagnosis of seronegative RA.

The present disclosure is based, in part, on the discovery that certain molecular biomarkers and/or clinical parameters are useful for predicting the likelihood of developing rheumatoid arthritis (RA), disease subtyping and classification (ACPA+ or ACPA−), and for predicting response to antirheumatic drugs. Accordingly, the present disclosure provides methods, systems, combinations of tests, and collections of results useful for predicting whether an individual will develop RA, the subtype of the RA, and their treatment response.

### Definitions

As used herein, the singular forms “a,” “an,” and “the” include plural references, unless the content clearly dictates otherwise, and are used interchangeably with “at least one” and “one or more.”

The term “about,” has the meaning reasonably ascribed to it by a person skilled in the art when used in conjunction with a stated numerical value or range, i.e. denoting somewhat more or somewhat less than the stated value or range, to within a range of ±20% of the stated value; ±15% of the stated value; ±10% of the stated value; ±5% of the stated value; ±4% of the stated value; ±3% of the stated value; ±2% of the stated value; 1% of the stated value; or ±any percentage between 1% and 20% of the stated value.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

The term “amount” or “level” as used herein refers to a quantity of a biomarker that is detectable or measurable in a biological sample and/or control. The quantity of a biomarker can be, for example, a quantity of polypeptide, the quantity of nucleic acid, or the quantity of a fragment or surrogate. The term can alternatively include combinations thereof. The term “amount” or “level” of a biomarker is a measurable feature of that biomarker.

As used herein, the term “molecular features” refers to features that are seen on a molecular level, including for example DNA, RNA, and proteins, genotypic data, phenotypic data, or the like.

The term “biomarker” refers to a biological molecule, or a fragment of a biological molecule, whose presence, level, or form, correlates with a particular biological event or state of interest, such that it is considered to be a “marker” of that event or state. To give but a few examples, in some embodiments, a biomarker may be or comprise a marker for a particular disease state, or for likelihood that a particular disease, disorder and/or condition may develop, occur, or reoccur. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, an amino acid, a hormone, an antibody, a cytokine, a chemokine, a growth factor, a steroid, a glycoprotein, a ribonucleoprotein, a lipoprotein, a small molecule, an inorganic agent (e.g., a metal or ion), together with their related metabolites, mutations, isoforms, variants, polymorphisms, modifications, fragments, subunits, degradation products, elements, and other analytes or sample-derived measures. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, plasma, serum, synovial fluid, etc). Biomarkers also encompass non-blood borne factors and non-analyte physiological markers of health status, and/or other factors or markers not measured from samples (e.g., biological samples such as bodily fluids), such as clinical parameters and traditional factors for clinical assessments. Biomarkers can also include any indices that are calculated and/or created mathematically. Biomarkers can also include combinations of any one or more of the foregoing measurements, including temporal trends and differences. Biomarkers can include but are not limited to the biomarkers described in Tables A-F herein.

TABLE A
Plasma proteins that can distinguish ACPA− RA from Controls
Differentially abundant proteins (Gene symbol with Somalogic barcode)

ABHD4_21780-15; ABLIM3_13578-98; ACADSB_17403-14; ACLY_12700-9;
ADAMTS6_6441-62; ADSL_5023-23; AGFG1_11681-8; AHSG_3581-53;
AKIRIN2_23280-9; ALG2_23654-6; ANKRD27_12445-50; ANKRD40_23367-8;
AP4M1_10076-1; APPL1_12825-18; ARF4_18408-26; ARID3C_25484-120;
ARL2BP_18407-36; ASAP3_24455-2; ATP5MF_11539-4; BAP18_21153-5;
BLVRB_17148-7; C2CD2L_23704-11; C5_2381-52; CA1_4969-2; CA3_3799-11;
CACYBP_12432-23; CAMK2A_3350-53; CAMK2D_3419-49; CAVIN1_24689-9;
CCL16_4913-78; CCM2_12347-29; CD2AP_21961-14; CD93_14136-234;
CERT1_13535-2; CERT1_13950-9; CFB_4129-72; CFD_13678-169; CFD_2946-52;
CFHR5_16055-3; CFHR5_7885-17; CFI_2567-5; CHAD_13460-4; CHRDL1_3362-61;
CLIC3_24693-5; CLIC5_12475-48; CLINT1_11659-31; CNOT1_13482-14; CNOT9_8975-
26; COL15A1_8974-172; COLEC11_4430-44; CPB2_3518-54; CSNK1G1_20130-144;
CTHRC1_15467-10; CTRB2_5648-28; CUL3_10045-47; CXorf38_24707-6;
CYLD_21765-10; DAPK3_24261-202; DERL1_13393-46; DFFA_21249-115;
DIAPH1_25083-26; DLG2_19620-16; DLGAP4_24436-23; DLK1_6373-54; DLK1_6496-
60; DLX4_11910-27; DMTN_23650-2; DNAJB11_7110-2; DNAJB4_18884-22;
DNAJC10_8297-8; DNMT3L_19142-39; DYNC1LI2_25053-1; EEF1A1_25256-23;
EIF1B_12701-1; EIF3J_13497-34; EIF4H_5885-55; EIF5A2_11355-10; ENO2_10339-48;
EPHA4_16288-17; EPN1_24255-38; EPS8L2_24891-54; ERC1_24983-119; ESM1_3805-
16; ETS2_12350-86; F10_4878-3; F9_4876-32; F9_5307-12; FAF2_9738-7;
FAM160B1_25081-6; FBXL5_12846-3; FCAMR_9568-289; FER_4220-39; FGL1_5581-
28; FKBP5_21577-35; FLII_12677-164; FMR1_7713-102; FN1_3434-34; FOXM1_10056-
5; FOXP4_24259-24; FSCN1_24226-30; G3BP2_9831-12; GATM_18188-12;
GIMAP4_24684-7; GLUD1_25902-27; GM2A_15441-6; GP1BB_21514-1; GPC1_8697-
38; GRB10_11358-15; GSN_16607-78; GSTK1_13474-40; GTPBP10_24712-6;
GZMA_13712-104; GZMA_3440-7; HBA1|HBB_4915-64; HBEGF_14094-29;
HGS_13644-30; HIF1A_13089-6; HMBS_11530-37; HOXA11_22375-15; HP_3054-3;
HP_7905-30; HS2ST1_21736-60; HSD17B4_23643-22; HSPA1A_14237-1;
IGFBP6_14088-38; IGFBP6_2686-67; IHH_19606-28; IKBKG_21976-4; IL18RAP_10457-
3; INPP4B_24427-33; KDM4C_25298-53; KIAA0040_14603-51; KLC1_12656-1;
KLK8_2834-54; KPNA1_19587-12; KPNA5_21235-11; LAMA4_6577-64; LPIN1_24469-
10; LRP11_15472-16; LTB4R_13477-65; LYN_3453-87; LYRM1_22496-21;
MAP2K5_22041-26; MARK3_9747-48; MCF2L_13934-3; MESD_15299-102;
METAP1_3210-1; MFAP4_5636-10; MICU2_6226-69; MITF_22383-21; MLX_23294-19;
MRPL10_22554-101; MSN_5009-11; MTRF1L_11134-30; MTSS2_25252-29;
MVB12B_23320-11; MYO6_9894-13; MYOC_16558-2; MYSM1_11536-9; MZT2A_24928-
20; NAMPT_5011-11; NAPG_17773-26; NBR1_7696-3; NCKIPSD_23677-30;
NEIL1_21802-53; NELL2_6022-57; NFE2L2_22510-6; NME1_5909-51; NOTCH3_5108-
72; NSMCE2_22517-106; NUDT16L1_12497-29; OAZ1_9893-27; OLFML3_8660-5;
OSBP_25055-56; PAGR1_23967-8; PAPOLG_8343-224; PCOLCE_11237-49;
PDGFC_13658-31; PGAM1_3896-5; PGAM2_15524-30; PGLYRP2_5601-2;
PGRMC1_7863-50; PGRMC2_10631-9; PID1_23291-18; PKLR_11203-97; PKP2_14067-
6; PLCD1_24909-40; PLEKHA1_12459-13; PMM2_17794-6; POLI_13536-56;
POU2AF1_21839-3; PPFIA1_13975-56; PPIC_18819-21; PPP1R9B_21991-79;
RAB32_17778-19; RAB3D_19379-154; RAF1_10001-7; RANBP3_14037-18;
RBPMS2_23300-3; RGMB_3331-8; RIPK2_8970-9; RNF215_9064-12; RRAS_17742-2;
S100A10_15318-75; S100A16_17836-17; SEPTIN11_12620-3; SERPINB13_18841-1;
SETD3_24260-4; SH3BP5_23662-10; SH3GL2_12499-108; SH3GL3_18318-98;
SHC1_16043-30; SHC1_5272-55; SIRT3_17495-141; SLBP_24648-8; SLITRK6_16916-
19; SMAP1_11649-3; SMTN_12546-1; SPARC_14110-200; SSPN_23275-9;
STAT3_10346-5; STIM1_8916-32; STX12_10418-36; STX6_10945-11; STX8_10903-50;
SYAP1_23409-83; TARBP2_12781-2; TBC1D13_21163-21; TBL2_11227-31;
TEC_16079-2; TFCP2L1_25104-10; TGFBI_3283-21; TLN2_14082-56; TMEM38B_9509-
4; TMOD1_12595-11; TMOD2_12853-112; TMUB1_25255-4; TOLLIP_13963-7;
TOR1AIP2_10553-8; TP53_6168-11; TPPP3_20444-12; TRAF1_17747-45;
TRAPPC2_20402-11; TRIM3_12573-80; TXNL4B_15316-262; UBE2S_17729-20;
UCN3_10756-34; USP19_21730-56; USP22_21757-49; USP25_9215-117; USP4_21763-
46; USP8_13450-49; UXS1_8258-22; VARS1_13083-18; VAV1_5275-28;
VCPIP1_23187-9; VPS4B_12668-7; VTI1A_7952-2; VTI1B_8963-8; WEE2_25038-5;
WNK3_5493-17; WWP1_11307-33; XPNPEP3_23661-61; XXYLT1_6375-75;
YY1_17337-1; ZC3H12A_23680-1; ZDHHC4_22834-21

TABLE B
Plasma metabolites that can distinguish ACPA− RA from Controls
Differentially abundant metabolites

X-12462; pyruvate; X-19438; sphingosine 1-phosphate; 3,5-dichloro-2,6-dihydroxybenzoic
acid; X-24295; 1-oleoyl-2-docosahexaenoyl-GPC (18:1/22:6); cys-gly, oxidized; sarcosine;
X-25343; X-15245; X-23276; X-23666; X-12104; 2′-deoxyuridine; alpha-ketoglutaramate;
3-bromo-5-chloro-2,6-dihydroxybenzoic acid; lactate; arginine; iminodiacetate (IDA); N-
acetyl-1-methylhistidine; X-24425; sphinganine-1-phosphate; 2′-O-methylcytidine;
deoxycarnitine; cortolone glucuronide (1); citrulline; X-23636; N-acetylarginine; N-acetyl-2-
aminooctanoate

TABLE C
Plasma autoantibodies that can distinguish ACPA− RA from Controls
Differentially abundant autoantibodies (anti-autoantigen target)

anti-CLK1; anti-FGFR1; anti-FAM46D; anti-LCP1; anti-FOXA3; anti-ULK4; anti-CNOT8;
anti-G6PD; anti-MAP2K1; anti-MED30; anti-HPCAL1; anti-ZNF398; anti-AXL_int; anti-
EGFR_int; anti-CDO1; anti-PBX3; anti-RPL18; anti-CALR; anti-DLX4; anti-TRIB3; anti-
KIT_int; anti-ZKSCAN1; anti-ELF2; anti-ALX1; anti-TWF2; anti-CDC25A; anti-EEF1A2;
anti-ZNF3; anti-IMP3; anti-SCP2; anti-ZSCAN9; anti-CEACAM1; anti-CKS1B; anti-HTR2B;
anti-NCK1; anti-CASP9; anti-RPS6KA5; anti-EIF4A2; anti-GRK5; anti-TBX5; anti-KRT14;
anti-CARHSP1; anti-DDX5; anti-PPP2CB; anti-CPA4; anti-PTGER3; anti-HSP90AA1; anti-
DCAF12; anti-LTA4H; anti-HTR1D; anti-SOX10; anti-RPS15A; anti-HEYL; anti-GATA3;
anti-PPP2R2C; anti-PRAME; anti-MX2; anti-CTNNBL1; anti-CDK8; anti-BMPR2; anti-
VPS45; anti-ACTB; anti-ETNK2; anti-BRD2; anti-RNASEL; anti-CHI3L1; anti-CPM; anti-
PLEKHA5; anti-PMEL; anti-TSG101; anti-GPATCH2; anti-CCR5; anti-EPHB3_int; anti-
RPL18A; anti-NDUFV3; anti-ZNF449; anti-IRAK4; anti-RUFY1; anti-ACVR2A; anti-
FGFR2_ext; anti-PPP3R1; anti-ISG20; anti-PAGE5; anti-SUMO2; anti-PDGFRA_ext; anti-
MORC1; anti-IL1B; anti-STK11; anti-TUBGCP3; anti-SLCO6A1; anti-WT1; anti-TMEM108;
anti-DCLK1; anti-CKM; anti-PIP4K2C; anti-JUP; anti-CTTN; anti-ODF2; anti-ARHGDIB;
anti-DSTYK; anti-DAPK2; anti-S100A9; anti-PDXK; anti-EHF; anti-SOCS2; anti-ZNF174;
anti-TDP2; anti-PKM; anti-MAP4; anti-CCDC36; anti-PIK3R1; anti-SOCS6; anti-BATF;
anti-MUSK; anti-MAGEB3; anti-PXK; anti-DDR1_ext; anti-TARDBP; anti-NFIL3; anti-IST1

TABLE D
Plasma proteins that can distinguish ACPA+ RA from Controls
Differentially abundant proteins (Gene symbol with Somalogic barcode)

MRPL50_22557-68; C9_13722-105; MAPK6_22858-3; CFB_4129-72; CFI_2567-5;
C9_3060-43; NAMPT_5011-11; TP53_6168-11; SERPINA4_14105-5; PGAM1_3896-5;
CFH_4159-130; C5_2381-52; SRL_10940-25; PZP_6580-29; MTPAP_9028-5;
CRP_4337-49; NELL2_6022-57; GPC4_18892-48; VARS1_13083-18; RTN4R_5105-2;
MAGED1_21392-15; PGAM2_15524-30; CGA|TSHB_3521-16; PRDM1_14197-2;
A2M_3708-62; ALDH7A1_21498-3; RBP4_7831-39; SIRT3_17495-141; MATN2_3325-2;
CHGB_8235-48; CXCL13_3487-32; DSC2_13126-52; GM2A_15441-6

TABLE E
Plasma metabolites that can distinguish ACPA+ RA from Controls
Differentially abundant metabolites

3,5-dichloro-2,6-dihydroxybenzoic acid; alpha-ketoglutaramate; hydroxyasparagine;
citrulline; 3-bromo-5-chloro-2,6-dihydroxybenzoic acid; N-acetylhistidine; pseudouridine;
N,N,N-trimethyl-alanylproline betaine (TMAP); gamma-glutamylcitrulline; cysteine; N-
acetylkynurenine (2); erythritol; perfluorohexanesulfonate (PFHxS); retinol (vitamin A); 5-
(galactosylhydroxy)-lysine; N-acetylserine; retinal; 5,6-dihydrouridine; N-palmitoyl-
sphingosine (d18:1/16:0); N6-succinyladenosine; N,N-dimethyl-pro-pro; caprate (10:0); X-
17010; N2,N5-diacetylornithine; gamma-glutamylglycine; branched-chain, straight-chain,
or cyclopropyl 10:1 fatty acid (1); 2′-deoxyuridine; 2,3-dihydroxy-5-methylthio-4-
pentenoate (DMTPA); X-23276; N-formylmethionine; glyco-beta-muricholate;
indolelactate; N-acetylvaline; ergothioneine; 1-ribosyl-imidazoleacetate; creatinine;
deoxycarnitine; 3-formylindole; oxindolylalanine; methionine sulfone; stearoyl-
arachidonoyl-glycerol (18:0/20:4) [2]; X-13007; histidine betaine (hercynine); S-
adenosylhomocysteine (SAH); 4-acetamidobutanoate; histidine; 1-methylguanidine; 3-
methoxytyramine sulfate; N-acetyltryptophan; sphingosine; stearoyl-arachidonoyl-glycerol
(18:0/20:4) [1]

TABLE F
Plasma autoantibodies that can distinguish ACPA+ RA from Controls
Differentially abundant autoantibodies (anti-autoantigen target)

anti-STK4; anti-CNOT8; anti-GAS2; anti-ECI2; anti-CMC4; anti-PTPN4; anti-COTL1; anti-
THUMPD1; anti-GNG11; anti-ERCC3; anti-HPCAL1; anti-AK3; anti-TXN2; anti-OVOL1;
anti-NMRAL1; anti-CDO1; anti-MAP2K1; anti-WT1; anti-SHPK; anti-RAB38; anti-DUSP4;
anti-CLK1; anti-SNCA; anti-MAP3K14; anti-CCNB1; anti-ELK3; anti-GRB7; anti-CDK9;
anti-BIRC5; anti-SSX1; anti-PRDM4; anti-S100A8; anti-NUAK2; anti-ANKRD45; anti-
NFX1; anti-BOLA3; anti-CYLD; anti-PAGE1; anti-VPS45; anti-LATS1; anti-RARB; anti-
ANXA2; anti-STAT5A; anti-ARNT; anti-ZNHIT3; anti-IRF5; anti-NFIL3; anti-TAGLN; anti-
PRKACB; anti-FOSL2; anti-SMAD9; anti-SBDS; anti-LCK; anti-HOOK1; anti-STAM; anti-
TFCP2L1; anti-CHMP3; anti-AURKA; anti-PBX1; anti-NRIP3; anti-PTP4A2; anti-MLF1;
anti-IL1RN; anti-PFN2; anti-TUBGCP3; anti-FOXN2; anti-STK26; anti-HSPD1; anti-
PAGE5; anti-STK10

As used herein, the term “panel” refers to a composition, such as an array or a collection, comprising one or more biomarkers. The number of biomarkers useful for a biomarker panel is based on the sensitivity and specificity value for the particular combination of biomarker values.

The term “antibody” refers to any immunoglobulin-like molecule that reversibly binds to another with the required selectivity. Thus, the term includes any such molecule that is capable of selectively binding to a biomarker of the present teachings. The term includes an immunoglobulin molecule capable of binding an epitope present on an antigen. The term is intended to encompass not only intact immunoglobulin molecules, such as monoclonal and polyclonal antibodies, but also antibody isotypes, recombinant antibodies, bi-specific antibodies, humanized antibodies, chimeric antibodies, anti-idiopathic (anti-ID) antibodies, single-chain antibodies, Fab fragments, F(ab′) fragments, fusion protein antibody fragments, immunoglobulin fragments, Fv fragments, single chain Fv fragments, and chimeras comprising an immunoglobulin sequence and any modifications of the foregoing that comprise an antigen recognition site of the required selectivity.

The term “autoantibody” as used herein refers to an antibody produced by an individual, where the antibody is directed against one or more ‘self’ antigens (e.g., antigens that are native to the individual, e.g., an antigen on a cell or tissue, or an endogenous peptide or protein). The present disclosure permits the detection of a variety of different autoantibodies, as demonstrated by the studies described herein.

“Autoimmune disease” encompasses any disease, as defined herein, resulting from an immune response against substances and tissues normally present in the body. Examples of suspected or known autoimmune diseases include rheumatoid arthritis, early rheumatoid arthritis, axial spondylarthrites, juvenile idiopathic arthritis, seronegative spondyloarthropathies, ankylosing spondylitis, psoriatic arthritis, antiphospholipid antibody syndrome, autoimmune hepatitis, Behçet's disease, bullous pemphigoid, coeliac disease, Crohn's disease, dermatomyositis, Goodpasture's syndrome, Graves' disease, Hashimoto's disease, idiopathic thrombocytopenia purpura, IgA nephropathy, Kawasaki disease, systemic lupus erythematosus, mixed connective tissue disease, multiple sclerosis, myasthenia gravis, polymyositis, primary biliary cirrhosis, psoriasis, scleroderma, Sjögren's syndrome, ulcerative colitis, vasculitis, Wegener's granulomatosis, temporal arteritis, Takayasu's arteritis, Henoch-Schonlein purpura, leucocytoclastic vasculitis, polyarteritis nodosa, Churg-Strauss Syndrome, and mixed cryoglobulinemic vasculitis.

The term “analyte” in the context of the present teachings can mean any substance to be measured, and can encompass biomarkers, markers, nucleic acids, electrolytes, metabolites, proteins, antibodies, sugars, carbohydrates, fats, lipids, cytokines, chemokines, growth factors, proteins, peptides, nucleic acids, oligonucleotides, metabolites, mutations, variants, polymorphisms, modifications, fragments, subunits, degradation products and other elements. For simplicity, standard gene symbols may be used throughout to refer not only to genes but also gene products/proteins, rather than using the standard protein symbol.

To “analyze” includes determining a value or set of values associated with a sample by measurement of analyte levels in the sample. “Analyze” may further comprise comparing the levels against constituent levels in a sample or set of samples from the same subject or other subject(s). The biomarkers of the present teachings can be analyzed by any of various conventional methods known in the art. Some such methods include but are not limited to: measuring serum protein or sugar or metabolite or other analyte level, measuring enzymatic activity, and measuring gene expression.

As used herein, the term “omics” refers to the collective technologies used to explore the roles, relationships, and actions of the various types of molecules that make up the cells of an organism. Generally, omics can include genomics, proteomics, transcriptomics, and/or metabolomics.

As used herein, the term “metabolite” refers to a low molecular compound (<1 kDa), smaller than most proteins, DNA and other macromolecules. Metabolites are the end products of cellular processes and their levels can be regarded as the ultimate measurable response of biological systems to physiological changes. Small changes in activity of proteins result in big changes in the biochemical reactions and their metabolites whose concentrations, fluxes and transport mechanisms are sensitive to diseases and drug intervention. Thus metabolomics-based biomarkers reflect aspects of the physiological state and provide diagnostic tools for clinical routines.

The term “metabolome” refers to the complete set of small-molecule metabolites (such as metabolic intermediates, hormones and other signaling molecules, and secondary metabolites) to be found within a biological sample, such as a single organism, at a given time under a given condition. The metabolome is dynamic and may change from second to second.

As used herein, “metabolomic profiling” refers to the determination of a metabolite (or preferably metabolites) in a biological sample.

As used herein, the term “cytokine” refers to small proteins (“5-20 kDa) that are important in cell signaling. Cytokine release has an effect on the behavior of cells around them. Cytokines are also involved in autocrine signaling, paracrine signaling and endocrine signaling as immunomodulating agents. The term “cytokine” includes chemokines, interferons, interleukins, lymphokines, and tumor necrosis factors. They are produced by macrophages, B lymphocytes, T lymphocytes as well as endothelial cells, fibroblasts, and various stromal cells. Cytokines modulate the balance between humoral and cell-based immune responses. There are both pro-inflammatory cytokines and anti-inflammatory cytokines. Pro-inflammatory cytokines include IL-1β, Interleukin 6 (IL-6), and TNF-α, which are involved in the process of pathological pain. Anti-inflammatory cytokines include IL4, IL-10, IL-13 and IFN-alpha. Cytokines are critical mediators that oversee and regulate immune and inflammatory responses via complex networks and and serve as biomarkers for many diseases.

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets. Also of use in the context of biomarkers are linear and non-linear equations and statistical classification analyses to determine the relationship between (a) levels of biomarkers detected in a subject sample and (b) the level of the respective subject's disease activity.

As used herein, the term “AUC” refers to the Area Under the Curve, for example, of a ROC Curve. That value can assess the merit of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for an ROC curve, such as, JMP™ or Analyse-It™. AUC can be used to compare the accuracy of the classification algorithm across the complete data range. Classification algorithms with greater AUC have, by definition, a greater capacity to classify unknowns correctly between the two groups of interest (disease and no disease). The classification algorithm may be the measure of a single molecule or as complex as the measure and integration of multiple molecules.

Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

As used herein, “obtaining” or “acquiring” (with regard to samples), may be any means by which a person of skill in the art possesses a sample by either “direct” or “indirect” means. Directly obtaining a sample means performing a process (e.g., performing a physical method such as extraction) to obtain a sample. Indirectly obtaining a sample refers to receiving the sample from another party or source (e.g., a third-party laboratory that directly takes the sample). Directly obtaining a sample includes performing a process that includes a physical change in a physical substance, e.g., a starting material such as blood, e.g., blood previously isolated from a patient. Thus, acquiring is used to mean collecting and/or removing a sample from a subject. Further, “obtaining” is also used to mean receiving the sample from another party that previously possessed the sample.

As used herein, “feature” refers to a measurable property or characteristic for subjects in the data set. Features include but are not limited to protein measurements and clinical factors.

The term “heat map” as used herein, refers to any graphical representation of data where the individual values contained in a matrix are represented as colors. Fractal maps and tree maps both often use a similar system of color-coding to represent the values taken by a variable in a hierarchy.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### Methods

### Detecting and Measuring Levels of Biomarkers in a Sample

As part of the present method, a panel of markers from an asymptomatic human subject may be measured. There are many methods known in the art for measuring either gene expression (e.g., mRNA) or the resulting gene products (e.g., polypeptides or proteins), metabolites, cytokines, autoantibodies or any other disclosed metabolites, that can be used in the present methods.

Suitable methods include chromatography (e.g., high-performance liquid chromatography (HPLC), gas chromatography (GC), liquid chromatography (LC)), mass spectrometry (e.g., MS, MS-MS), NMR, enzymatic or biochemical reactions, immunoassay, and combinations thereof. For example, mass spectrometry can be combined with chromatographic methods, such as liquid chromatography (LC), gas chromatography (GC), or electrophoresis to separate the metabolite being measured from other components in the biological sample. See, e.g., Hyotylainen (2012) Expert Rev. Mol. Diagn. 12(5):527-538; Beckonert et al. (2007) Nat. Protoc. 2(11):2692-2703; O'Connell (2012) Bioanalysis 4(4):431-451; and Eckhart et al. (2012) Clin. Transl. Sci. 5(3):285-288; the disclosures of which are herein incorporated by reference. Alternatively, analytes can be measured with biochemical or enzymatic assays.

Immunoassays based on the use of antibodies that specifically recognize a biomarker may be used for measurement of biomarker levels. Such assays include (but are not limited to) enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975).

Antibodies may be used in diagnostic assays to detect the presence or for quantification of the biomarkers in a biological sample. Such a diagnostic assay may comprise at least two steps; (i) contacting a biological sample with the antibody, wherein the sample is blood or plasma, a microchip (e.g., See Kraly et al. (2009) Anal Chim Acta 653(1):23-35), or a chromatography column with bound biomarkers, etc.; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

In various embodiments, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one-dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the disclosure, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

In a number of embodiments, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

After preparation, biomarkers in a sample are typically captured on a substrate for detection. Traditional substrates include antibody-coated 96-well plates or nitrocellulose membranes that are subsequently probed for the presence of biomarkers. Alternatively, metabolite-binding molecules attached to microspheres, microparticles, microbeads, beads, or other particles can be used for capture and detection of biomarkers. The metabolite-binding molecules may be antibodies, peptides, peptoids, aptamers, small molecule ligands or other metabolite-binding capture agents attached to the surface of particles. Each metabolite-binding molecule may comprise a “unique detectable label,” which is uniquely coded such that it may be distinguished from other detectable labels attached to other metabolite-binding molecules to allow detection of biomarkers in multiplex assays. Examples include, but are not limited to, color-coded microspheres with known fluorescent light intensities (see e.g., microspheres with xMAP technology produced by Luminex (Austin, TX); microspheres containing quantum dot nanocrystals, for example, having different ratios and combinations of quantum dot colors (e.g., Qdot nanocrystals produced by Life Technologies (Carlsbad, CA); glass coated metal nanoparticles (see e.g., SERS nanotags produced by Nanoplex Technologies, Inc. (Mountain View, CA); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, CA); chemiluminescent dyes, combinations of dye compounds; and beads of detectably different sizes. See, e.g., U.S. Pat. Nos. 5,981,180, 7,445,844, 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

Mass spectrometry, and particularly SELDI mass spectrometry, is useful for detection of biomarkers. Laser desorption time-of-flight mass spectrometer can be used in embodiments of the disclosure. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting biomarkers. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the disclosure.

### Methods of Treatment and Prevention

In other embodiments, the present disclosure relates to a method of treating a subject afflicted with rheumatoid arthritis (RA), wherein the term “rheumatoid arthritis” includes juvenile rheumatoid arthritis, juvenile idiopathic arthritis, ankylosing spondylitis disease, Sjögren's syndrome, psoriatic arthritis. The term rheumatoid arthritis also encompasses subgroups rheumatoid arthritis including seropositive RA, seronegative RA, ACPA+ RA, ACPA− RA, etc.

“Treatment” of a patient or a subject refers to both therapeutic treatment and prophylactic or preventative measures. The terms “effective amount” or “therapeutically effective” refer to an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder and/or condition. For example, such an effective amount can result in any one or more of reducing the signs or symptoms of RA (e.g. achieving ACR20), reducing disease activity (e.g. Disease Activity Score, DAS20), slowing the progression of structural joint damage or improving physical function. In some embodiments, a therapeutically effective amount is administered in a single dose; in some embodiments, multiple unit doses are required to deliver a therapeutically effective amount.

Accordingly, in some embodiments, the present disclosure provides a method of treating a subject afflicted with rheumatoid arthritis (RA), said method comprising the steps of diagnosing the subject with rheumatoid arthritis (RA), comprising:
(a) obtaining a blood sample from the subject; and
(b) determining the amount of a plurality of biomolecules comprising plasma proteins, plasma metabolites and plasma autoantibodies in the blood sample; wherein
(i) the amount of at least 25 plasma proteins from Table A, and/or at least 25 plasma metabolites from Table B, and/or at least 25 plasma autoantibodies from Table C, or a combination of at least 50 biomolecules from Tables A, B and C, is higher in the blood sample relative to an amount of the biomolecules in a control sample from a subject without RA and indicates the subject has seronegative RA (ACPA− RA); or
(ii) the amount of at least 25 plasma proteins from Table D, and/or at least 25 plasma metabolites from Table E, and/or at least 25 plasma autoantibodies from Table F, or a combination of at least 50 biomolecules from Tables D, E and F, is higher in the blood sample relative to an amount of the biomolecules in a control sample from a subject without RA indicates the subject has seropositive RA (ACPA+ RA); and
(c) administering at least one therapy to the subject.

In some embodiments, the present disclosure provides a method treating a subject afflicted with seronegative RA (ACPA− RA), said method comprising the steps of diagnosing the subject with seronegative RA (ACPA− RA), comprising:
(a) obtaining a blood sample from the subject; and
(b) determining the amount of biomolecules comprising plasma proteins, plasma metabolites and plasma autoantibodies in the blood sample;
(i) wherein the amount of at least 50 biomolecules from Tables A, B and C, is higher in the blood sample relative to an amount of the biomolecules in a control sample from a subject without RA and indicates the subject has seronegative RA (ACPA− RA); and
(c) administering at least one therapy to the subject.

In another embodiment, the present disclosure provides a method of of treating a subject afflicted with seropositive RA (ACPA+ RA), said method comprising the steps of diagnosing the subject with seropositive RA (ACPA+ RA), comprising:
(a) obtaining a blood sample from the subject; and
(b) determining the amount of biomolecules comprising plasma proteins, plasma metabolites and plasma autoantibodies in the blood sample;
wherein the amount of at least 50 biomolecules from Tables D, E and F, is higher in the blood sample relative to an amount of the biomolecules in a control sample from a subject without RA indicates the subject has seropositive RA (ACPA+ RA); and
(c) administering at least one therapy to the subject.

In some embodiments, the determining the amount of biomolecules in step (b) produces a multi-omic data set, wherein the multi-omic data set is processed through a trained machine learning model, and wherein the processed multi-omic data set provides a RA phenotype classification.

In yet another embodiment, the present disclosure relates to a method of treating a subject afflicted with seronegative RA (ACPA− RA), said method comprising the steps of diagnosing the subject with seronegative RA (ACPA− RA), comprising:
(a) obtaining a blood sample from the subject; and
(b) administering at least one therapy to the subject.

In yet another embodiment, the present disclosure provides a method of treating a subject with rheumatoid arthritis (RA), said method comprising the steps of:
(a) diagnosing a subject with rheumatoid arthritis (RA), said method comprising the steps of preparing a RA phenotype classification network comprising:
(a) receiving multi-omic data from a blood sample from a subject, wherein the multi-omic data includes proteomic, metabolomic, and autoantibody profiles;
(b) processing the received multi-omic data through a trained machine learning model;
(c) classifying the subject's RA phenotype based on the processed multi-omic data; and
(d) outputting the classification result; and wherein:
(i) the classification is ACPA− RA when the amount of at least 25 plasma proteins from Table A, and/or at least 25 plasma metabolites from Table B, and/or at least 25 plasma autoantibodies from Table C, or a combination of at least 50 biomolecules from Tables A, B and C is detected; or
(ii) the classification is ACPA+ RA when the amount of at least 25 plasma proteins from Table D, and/or at least 25 plasma metabolites from Table E, and/or at least 25 plasma autoantibodies from Table F, or a combination of at least 50 biomolecules from Tables D, E and F is detected; and
(e) administering at least one therapy to the subject. In some embodiments, the method comprises administering an effective amount of a disease modifying anti-rheumatic drug (DMARD). The members of this category of drugs are known to the person skilled in the art. DMARDs include both biological (or “biologic”) and non-biological (or “non-biologic”) drugs.

Biological DMARDs include etanercept, adalimumab, infliximab, certolizumab pegol, and golimumab, which are all part of a class of drugs called tumor necrosis factor (TNF) inhibitors, and a variety of other agents with different targets, including anakinra, abatacept, rituximab, and tocilizumab.

Non-biological DMARDs include methotrexate, aspirin, hydroxychloroquine and leflunomide. Another common non-biological DMARDs is sulfasalazine. Less frequently used non-biological DMARDs include gold salts, azathioprine, and cyclosporine. The DMARD may therefore be selected from the group consisting of methotrexate, aspirin, hydroxychloroquine, leflunomide, sulfasalazine, gold salts, azathioprine, and cyclosporine. The DMARD may be selected from the group consisting of methotrexate, aspirin, hydroxychloroquine, leflunomide and sulfasalazine. The DMARD may be selected from the group consisting of methotrexate, aspirin, hydroxychloroquine and leflunomide. The DMARD may be methotrexate.

In other embodiments, the therapy comprises a different drug class, wherein the different drug class comprises son-steroidal anti-inflammatory drugs (NSAIDs) or glucocorticoids.

The non-steroidal anti-inflammatory agents suitable for use in the methods of treatment for arthritis described herein include all non-steroidal anti-inflammatory drugs (NSAIDs) used to treat undesirable inflammation of body tissues. Suitable NSAIDs for use in the treatment described herein include, but are not limited to, indole-based anti-inflammtory agents (including among others, indomethacin, indoxole and the like); salicylate-based anti-inflammatory agents (including among others, aspirin and the like); phenylacetic acid-based anti-inflammatory drugs (including, among others, fenoprofen, ketoprofen, MK-830 and the like); pyrazolidine-based anti-inflammatory agents (including, among others, phenylbutazone, oxyphenbutazone, and the like); and p-(isobutylphenyl) acetic acid-based anti-inflammatory agents (including, among others, buprofen, ibufenac, and the like).

NSAIDS preferred for use in the methods of treatment described herein include, but are not limited to, salicylates, indomethacin, flurbiprofen, diclofenac, ciclofenac, celecoxib, naproxen, piroxicam, tebufelone, and ibuprofen. Other NSAIDs suitable for use herein include, but are not limited to, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, and tolmetin.

Suitable glucocorticoids for treatment in accord with the methods described herein include but are not limited to prednisone, prednisolone, methylprednisolone, budesonide, dexamethasone, fludrocortisone, fluocortolone, cloprednole, deflazacort, and triamcinolone, and the corresponding salts and esters thereof.

In various embodiments, the methods disclosed herein can be used to monitor the efficacy of a treatment in an RA patient, wherein the methods further comprise obtaining a sample before and after treatment and determining the presence and/or levels of biomolecules over time. In some embodiments, the methods further comprise identifying a patient likely to benefit from a therapy for RA. In still other embodiments, the methods further comprise distinguishing a patient with ACPA− versus ACPA+ RA.

### EXAMPLES

### Example 1: Materials and Methods

### Study Population, Subject Enrollment, and Plasma Sample Collection

The study population consisted of patients with RA attending the outpatient practice of the Division of Rheumatology at Mayo Clinic in Rochester, MN, USA. Eligibility required patients to be adults 18 years of age or older with a clinical diagnosis of RA by a rheumatologist, fulfilling the American College of Rheumatology/European League Against Rheumatism 2010 revised classification criteria for RA (4). Patients were excluded if they did not comprehend English, were unable to provide written informed consent, or were members of a vulnerable population (e.g., incarcerated subjects). RA was categorized into either ACPA− or ACPA+ RA subgroups based on the titer of anti-CCP antibodies detected by the Quanta Lite CCP3 lgG enzyme-linked immunosorbent assay (INOVA Diagnostics; negative, <20.0 IU/mL). Subjects in the healthy control group were reported as not having any overt disease or adverse symptoms at the time of sample collection. Demographic and clinical data, including the numbers of tender and swollen joints, patient and evaluator global assessments, CRP (mg/L), BMI (kg/m2), smoking history, and results for RF (IU/mL) and anti-CCP antibodies were collected from the electronic medical records.

Plasma samples from patients with RA were stored in the ongoing Mayo Clinic Rheumatology Biobank. This biorepository was created for long-term storage of diverse biological samples (e.g., serum, plasma, stool, white blood cells) from patients for use in research. In addition, plasma samples from healthy donors participating in the Mayo Clinic Biobank were used as controls. This study was approved by the Mayo Clinic Institutional Review Board (No. 21-002409 and No. 22-001198) in accordance with the Declaration of Helsinki. All methods and procedures were performed in accordance with the Mayo Clinic Institutional Review Board guidelines and regulations.

### Proteomic Profiling

Plasma proteins were measured with SomaLogic's SomaScan Assay version 4 (56), which simultaneously targets over 7,000 human proteins including cytokines, growth factors, proteases, and hormones. This platform relies upon protein-capture SOMAmer (Slow Offrate Modified Aptamer) reagents. SOMAmers are based on single-stranded, chemically modified nucleic acids, and are designed to optimize high affinity, slow off-rate, and high specificity to target proteins. In brief, the multiplexed, aptamer-based assay measures the relative binding of target proteins to aptamers in relative fluorescence units (RFUs). After protein concentrations were converted into corresponding DNA aptamer concentrations, abundance levels of proteins were quantified with a DNA microarray.

Data standardization, comprising normalization, plate scaling, and calibration, was performed on the raw assay data to remove systematic biases after microarray feature aggregation. Global reference standards were established for procedures with controls on each plate (i.e., run). Individual, quality control (QC), and calibrator samples were normalized and calibrated to the established global reference standards. In addition, SOMAmer reagents that represent control or non-human analytes were removed, resulting in 7,273 proteins for further analysis. Of note, proteins having the same name but with multiple barcodes (i.e., SeqID) were considered as separate features.

### Metabolomic Profiling

Metabolic profiles were measured by ultra-high-performance liquid chromatography-tandem mass spectrometry (UPLC-MS/MS) using the untargeted metabolomics Discovery HD4TM platform from Metabolon Inc (Durham, NC). Statistical analyses on untargeted metabolomic data were performed using scaled imputed data provided by Metabolon. Briefly, the raw data were normalized to account for inter-day variation, which is a result of UPLC-MS/MS runs over multiple days. The peak intensities were then rescaled to set each metabolite's median equal to 1. Missing values were then imputed with the minimum observed value of the metabolite across all samples, yielding the scaled imputed data. In addition, metabolites with missing values in over 20% of the entire samples were removed, resulting in 1,061 metabolites remaining for further analysis.

### Autoantibody Profiling

Sengenics' immunome protein microarray platform was used to quantitatively profile over 1,600 IgG autoantibodies in plasma samples along with six pooled normal sera (Sengenics internal QC samples). In brief, the microarray consists of autoantigen proteins (as autoantibody targets) representing various functional categories, such as cancer-associated antigens, transcription factors, kinases, and those involved in inflammation and cell signaling. The autoantigen panel features full-length, correctly folded, native (i.e., non-post-translationally modified) proteins immobilized through a proprietary biotin carboxyl carrier protein (BCCP) onto its hydrogel-coated array surface. The conformation of the epitopes is preserved, allowing highly specific and reproducible detection of autoantibodies.

Autoantibody abundance was quantified using the median intensities of all the pixels within each probed spot of the microarray. Automatic extraction and quantification of pixels in each spot were performed using GenePix Pro 7 software. GenePix Results (.GPR) file was generated for each slide which contains the information for each spot (e.g., protein ID, protein name, foreground intensities, background intensities). Next, probes that were related to the QC process or had a high rate (>20%) of zeros were removed. Finally, raw RFUs of 1,610 autoantibodies were quantile normalized for further analysis.

### Identification of Phenotype-Associated Omics Features.

Omics features (i.e., proteins, metabolites, autoantibodies) associated with a clinical phenotype (i.e., study group) were identified using logistic regression analysis coupled with effect size (Cohen's d) determination. These analyses were conducted across three pairs of phenotype comparisons: ACPA− RA vs. controls, ACPA+ RA vs. controls, and ACPA+ RA vs. ACPA− RA. To mitigate potential confounding effects, logistic regression models were adjusted for sex, age, BMI, smoking history, use of prednisone, bDMARDs, and csDMARDs.

The logistic regression model for each omic feature was defined by Eqn. 1 below where, p is the probability of being equal to a certain phenotype, X is the vector of predictor variables (encompassing feature abundance and the potential confounders), and β is the vector of coefficients. A feature was considered to be associated with the phenotype (i.e., differentially abundant) if its corresponding coefficient in the logistic regression model was statistically significant (P<0.05) and its effect size was above medium (i.e., |d|>0.5) for proteins and metabolites, or above small (i.e., |d|>0.2) for autoantibodies.

$$log ( \frac{p}{1 - p} ) = {X}^{T} β \qquad Eqn . 1$$

For clarification, the use of bDMARDs refers to the prescription use of any of the following: abatacept, adalimumab, certolizumab, etanercept, infliximab, rituximab, or tocilizumab. Similarly, csDMARDs refers to hydroxychloroquine, leflunomide, methotrexate, or sulfasalazine.

### Functional Enrichment of Proteins

For a set of proteins, enriched functions defined by Gene Ontology biological process (GOTERM_BP_FAT) annotations were identified using DAVID (version 6.8) (34). Enrichment of a biological process was deemed significant for P-values less than 0.05, determined by a modified one-tailed Fisher's exact test.

### Construction of the Phenotype-Centric Multi-Omics Network

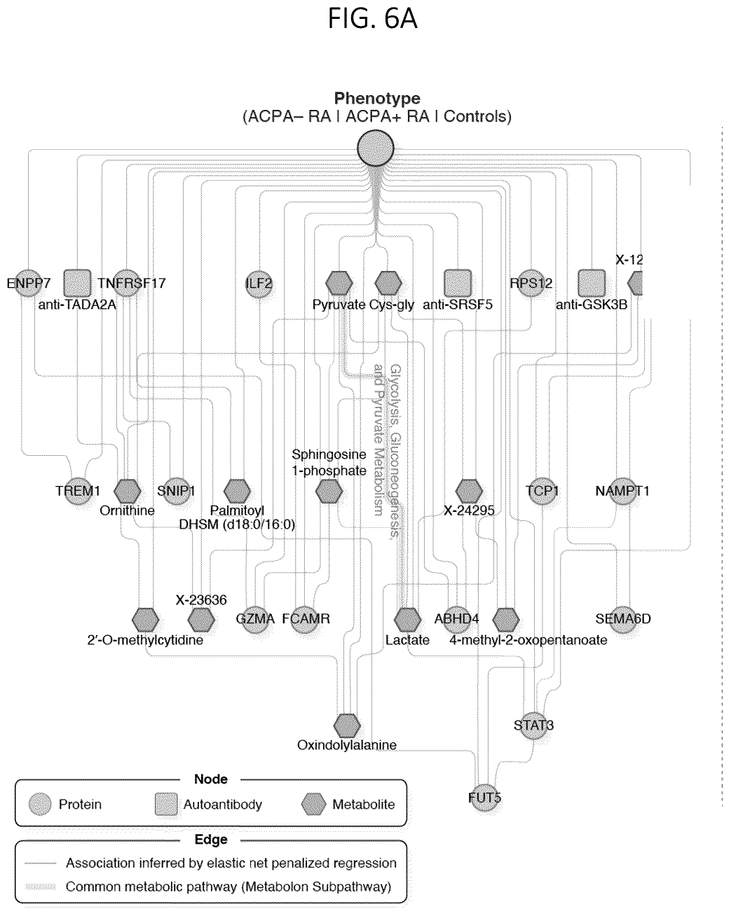

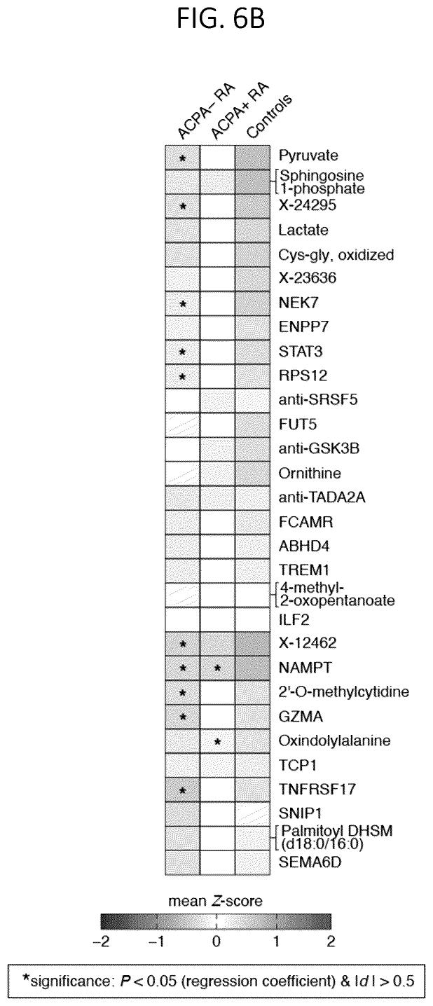

The phenotype-centric multi-omics network was constructed using a three-pronged approach: network inference, network diffusion, and subnetwork identification. In brief, elastic net regularization was used to infer a network that captures relationships (i.e., edges) between 9,949 features (i.e., nodes) across all 120 plasma samples. These features spanned proteomics, metabolomics, autoantibodies, clinical phenotype (i.e., ACPA− RA, ACPA+ RA, and healthy controls), and demographics, covering data from all samples across the three study groups. Subsequently, a random walk with restart (RWR) algorithm was applied on the inferred network to prioritize the selection of features most closely associated with the phenotype. The resulting subnetwork, characterized by only the subset of selected features, delineates the features most closely associated with (and thereby most predictive of) the phenotypes (FIG. 6). The following sections provide more details into the methodology.

### Inferring a Multi-Omics Network Using Elastic Net

Elastic net regularization is a combination of L1 and L2 regularizations, and is effective when p>>n, i.e., datasets where the number of features (p) significantly exceeds the number of samples (n) (57). In the present approach, each feature within the dataset was treated as a response variable in turn, and elastic net regularization was applied to identify predictor variables (i.e., all other features) that exhibited non-zero coefficients. Predictors with non-zero coefficients were considered to be associated with the response variable. An undirected graph was then constructed for each feature, where both response and predictor variables were represented as nodes, and edges indicated connections between the response variable and its predictors with non-zero coefficients. This process was repeated for each feature, resulting in 9,949 individual undirected graphs. Finally, these 9,949 graph models were merged to formulate a single, all-encompassing multi-omics network. The elastic net's loss function used in this analysis is defined by Eqn. 2 where n is the number of samples (1≤i≤n; n=120), p is the total number of features (1≤j≤p; p=9,948), y is the response variable, x represents the predictors with the collection of x excluding y. The hyperparameters λ₁ and λ₂ satisfy λ₁+μ₂=1, while the ratio between L1 regularization and L2 regularization falls within 0<λ₁:λ₂<1. The elastic net was implemented using R package “glmnet” (v4.1.1). Hyperparameters of the elastic net model were estimated using 10-fold cross-validation, with the optimal values chosen based on the model's performance in cross-validation. Selection of the best model was guided by the criterion of minimizing the root mean square error.

$${argmin}_{β} {\sum }_{i = 1}^{n} ( {y}_{i} - {β}_{0} - {\sum }_{j = 1}^{p} {β}_{j} {x}_{ij} ) + {λ}_{1} {\sum }_{j = 1}^{p} \vert {β}_{j} \vert + {λ}_{2} {\sum }_{j = 1}^{p} {β}_{j}^{2} \qquad Eqn . 2$$

Network Diffusion Using Random Walk with Restart.

Random walk with restart (RWR) was used to perform network diffusion on the previously inferred multi-omics network, aiming to identify a phenotype-centric multi-omics network. RWR, widely recognized as a guilt-by-association method, facilitates the exploration of a network's topology based on the premise that functionally similar nodes are often in close proximity to each other within networks (58). The R package “diffusr”, an implementation of the Markov random walk, was used to simulate network diffusion, as described by Eqn. 3 where p⁰ is the vector of initialized nodes, t is a time step, p^{t} is the vector at the current time step, p^{t+1} is the vector at the subsequent time step, A′ is a column-normalized version of the adjacency matrix A, and r is the restart rate. Elements of p⁰ are initialized as 1 or 0 to signify the seed node (i.e., sample phenotype) or all other features, respectively; and normalized to ensure the sum of the elements in p⁰ equals 1. For calculation simplicity, the adjacency matrix A only consists of 0 or 1 so that it represents a graph without weighted edges.

$${p}^{t + 1} = ( 1 - r ) {A}^{′} {p}^{t} + {rp}^{0} \qquad Eqn . 3$$

The network diffusion process was conducted using the default options of the “diffusr” R package, where the restart rate r (i.e., the probability of the random walker returning to the seed node in the next step of the walk) is set to 0.5, and the diffusion is terminated when the L1 norm difference between pₜ and p^{t+1} falls below 1.0×10⁻⁴. Upon completion, the nodes were assigned a “relevance score” reflecting the probability of the random walker being present at the corresponding node. These relevance scores were utilized to rank the network features, with higher scores indicating a stronger association with the sample phenotype. Through this approach, RWR propagates “importance” throughout the network, highlighting features closest to the seed node. Thus, network diffusion effectively prioritizes informative features that might otherwise be masked by less relevant neighbors.

Selection of Features Associated with Clinical Phenotype.

Following the RWR-mediated network diffusion, a relevance score was assigned to each feature reflecting its association with the sample phenotype. These scores were then ranked in descending order to create a hierarchy of feature importance. The top N features (e.g., the top 10, top 20, and so on up to all features) were selected from these ranked scores to construct a phenotype-centric network. (This network is termed “phenotype-centric” because its construction starts with the phenotype or study group as the seed node, around which the network is built.) The resulting network is a refined subnetwork, originally derived from the broader multi-omic network inferred by elastic net. This subnetwork is composed solely of nodes representing the top N features most strongly associated with the sample phenotype. Of note, the nodes within this phenotype-centric subnetwork are later used as predictors for training a random forest classifier.

Classification Performance of Features from the Phenotype-Centric Multi-Omics Network.

A 5-fold cross-validation scheme was performed to evaluate the classification performance of multi-omics features from the aforementioned phenotype-centric network. This evaluation was conducted on the plasma multi-omic profiles from the three study groups (ACPA− RA (n=40), ACPA+ RA (n=40), and controls (n=40)), with the aim of measuring the accuracy, sensitivity, specificity, positive predictive value, negative predictive value, and Matthew's correlation coefficient for clinical phenotype classification.

For each cross-validation fold, the dataset was divided into two segments: a training set comprising 96 plasma samples (32 from each group) and a test set with 24 samples (8 from each group). The phenotype-centric multi-omics network, which was derived from the training samples, was utilized as a template for selecting features, where each node was treated as a potential feature. The top N features were selected from this template network (e.g., the top 10, top 20, and so forth up to all features) for training a random forest classifier (seed=“123”). The classifier was tasked with predicting the phenotype, e.g., differentiating between ACPA− RA vs. controls, and ACPA+ RA vs. controls, on a balanced test set comprising 16 samples (8 from each group). Furthermore, the classifier's ability to distinguish between RA (combining both RA subgroups) and controls was tested on a test set of 24 samples (8 ACPA− RA, 8 ACPA+ RA, and 8 controls).

Evaluating ACPA− Vs. ACPA+ RA Classification Model Performance Using Two Independent Plasma Metabolomic Datasets.

Two independent validation datasets were utilized to evaluate the classifier's ability to distinguish between ACPA− RA and ACPA+ RA. The first dataset (Independent dataset #1) was obtained from the Mayo Clinic Early Arthritis Cohort Study (IRB no. 18-006677), which included plasma samples from 28 ACPA+ and 9 ACPA− RA patients who were newly diagnosed (i.e., disease duration ≤6 months and DMARD treatment-naïve). The second dataset (Independent dataset #2) was obtained from a previously published study (28), which consisted of plasma samples from 103 ACPA+ and 28 ACPA− RA patients under various treatment conditions. All samples from both cohorts were processed using UPLC-MS/MS on Metabolon Inc.'s Discovery HD4TM platform. Metabolomic measurements were scaled using the same strategy as in this study.

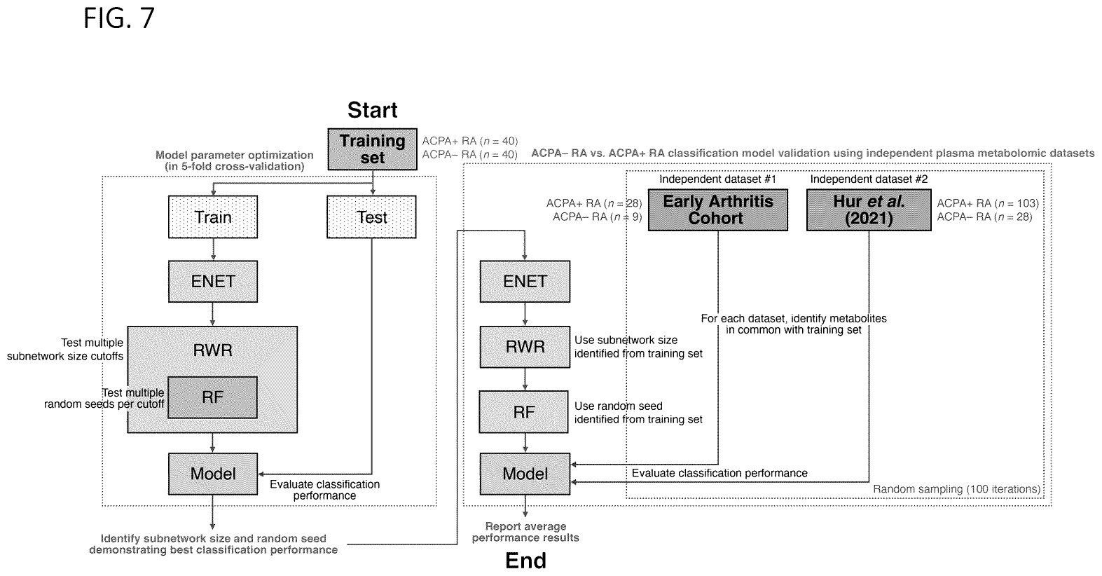

Network inference and network diffusion-based feature selection were used to identify metabolites from the training dataset of 80 samples (40 ACPA− and 40 ACPA+ RA), focusing on an optimized subnetwork size determined through 5-fold cross-validation. Metabolites absent in the independent datasets-likely due to data preprocessing discrepancies, quality control issues, or updates in the profiling platform's library-were excluded. A random forest classifier was trained with the retained features and tested on the two independent datasets (FIG. 7).

### Example 2

### Study Design and Participant Characteristics

A retrospective, observational cohort study design was employed. The study included a total of 120 participants comprising three study groups: patients with ACPA− RA (n=40), patients with ACPA+ RA (n=40), and healthy controls (n=40). Table 1 provides the demographic and clinical characteristics of the study participants.

TABLE 1
Demographic and clinical characteristics of study participants.

| | ACPA− RA | ACPA+ RA | Controls | P- |
|---|---|---|---|---|
| | (n = 40) | (n = 40) | (n = 40) | value* |
| Sex (Female/Male) | 28/12 | 29/11 | 28/12 | 1.0 |
| Age (Years) | | | | |
| Mean ± SD | 59.1 ± 10.5 | 56.8 ± 10.4 | 59.1 ± 10.5 | 0.45 |
| [Q1, Q3] | [55.0, 65.0] | [50.5, 64.3] | [55.0, 65.0] | |
| Range (min-max) | 32.0-76.0 | 35.0-74.0 | 32.0-76.0 | |
| Race (n, %) | | | | |
| White | 40 (100%) | 40 (100%) | 40 (100%) | 1.0 |
| Disease | | | | |
| duration (Years) | | | | |
| Mean ± SD | 2.6 + 2.8 | 2.8 + 2.6 | N/A | 0.66 |
| [Q1, Q3] | [0.1, 3.9] | [0.1, 4.5] | | |
| Range (min-max) | 0.1-9.5 | 0.0-9.3 | | |
| BMI | | | | |
| Mean ± SD | 30.7 ± 8.1 | 27.7 ± 5.6 | 30.2 ± 8.3 | 0.27 |
| [Q1, Q3] | [25.4, 34.0] | [25.2, 30.4] | [23.7, 34.3] | |
| Range (min-max) | 19.4-58.2 | 18.0-43.5 | 18.3-51.6 | |
| Smoking | | | | |
| history (n) | | | | |
| Current | 2 | 1 | 4 | 0.29 |
| Never/Former | 38 | 39 | 32 | |
| Unknown | 0 | 0 | 4 | |
| ESR (mm/hr) | | | | |
| Mean ± SD | 13.1 ± 15.9 | 13.3 ± 12.8 | N/A | 0.68 |
| [Q1, Q3] | [3.8, 16.3] | [4.0, 20.5] | | |
| Range (min-max) | 1.0-73.0 | 0.0-42.0 | | |
| Unknown | 0 | 2 | | |
| CRP (mg/L) | | | | |
| Mean ± SD | 15.3 ± 26.1 | 6.8 ± 9.4 | N/A | 0.09 |
| [Q1, Q3] | [2.9, 10.9] | [2.9, 4.9] | | |
| Range (min-max) | 2.9-113.5 | 2.9-54.0 | | |
| Unknown | 0 | 1 | | |
| RF (Yes/No) | 14/26 | 28/12 | N/A | 0.003 |
| DAS28-CRP | | | | |
| Mean ± SD | 3.9 ± 1.7 | 3.1 ± 1.4 | N/A | 0.07 |
| [Q1, Q3] | [2.4, 5.1] | [1.7, 4.2] | | |
| Range (min-max) | 1.5-7.5 | 1.5-6.4 | | |
| Unknown | 4 | 3 | | |
| Treatment (n, %) | | | | |
| Methotrexate | 19 (48%) | 22 (55%) | N/A | 0.65 |
| Prednisone | 12 (30%) | 8 (20%) | | 0.44 |
| TNFi- | 3 (8%) | 8 (20%) | | 0.19 |
| bDMARDs^{α} | | | | |
| Non-TNFi- | 4 (10%) | 2 (5%) | | 0.68 |
| bDMARDs^{β} | | | | |
| Non-MTX | 10 (25%) | 15 (38%) | | 0.33 |
| csDMARDs^{λ} | | | | |

ACPA− RA, anti-citrullinated protein antibody-negative rheumatoid arthritis;
ACPA+ RA, anti-citrullinated protein antibody-positive rheumatoid arthritis;
Q1/Q3, lower/upper quartile of the interquartile range;
ESR, erythrocyte sedimentation rate;
CRP, C-reactive protein;
RF, rheumatoid factor;
DAS28-CRP, Disease Activity Score 28 using C-reactive protein;
N/A, not available;
^{α}adalimumab, certolizumab, and etanercept;
^{β}abatacept, rituximab, and tocilizumab;
^{λ}hydroxychloroquine, leflunomide, and sulfasalazine;
*P-values for categorical and continuous variables were obtained using the Fisher's exact test and Kruskal-Wallis test, respectively.

All three study groups were matched based on subjects' age, body mass index (BMI), race (White), sex, and smoking history. At the time of plasma sample collection, all RA patients had established disease and had a mean age of 57.9 years (range: 32-76 years). The disease activity of the patients varied from remission to high disease activity, with a mean Disease Activity Score 28 using C-reactive protein (DAS28-CRP) (25, 26) of 3.5 (range: 1.5-7.5). Subsets of patients were on treatment with methotrexate (MTX, 51% or 41 of 80), prednisone (25% or 20 of 80), tumor necrosis factor inhibitor biologic disease-modifying anti-rheumatic drugs (TNFi-bDMARDs) (14% or 11 of 80), non-TNFi-bDMARDs (8% or 6 of 80), or non-MTX conventional synthetic disease-modifying anti-rheumatic drugs (non-MTX csDMARDs) (31% or 25 of 80).

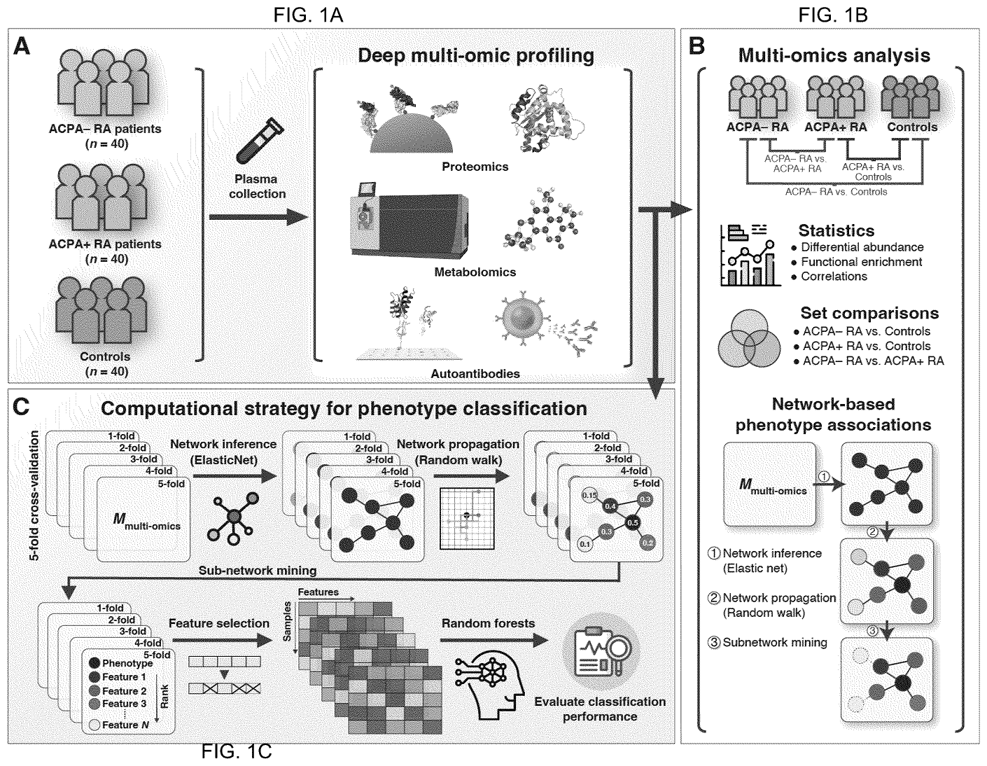

An overview of the study design is presented in FIG. 1. Deep (i.e., high-throughput, comprehensive) multi-omic measurements were conducted on plasma samples from the 120 study participants. The SomaScan Assay by SomaLogic (Boulder, CO, USA) was utilized for proteomic profiling, the Discovery HD4 platform by Metabolon (Durham, NC, USA) for metabolomic profiling, and the Sengenics Immunome Protein Microarray (Singapore) to quantify IgG autoantibodies (FIG. 1A). The associations between 9,944 biomolecular omic features (i.e., 7,273 proteins, 1,061 metabolites, 1,610 autoantibodies) and the three different study groups were explored using statistical analyses, set comparisons, and network inference techniques (FIG. 1B). Lastly, a machine learning approach that combined network inference and supervised classification on the multi-omic datasets was applied to develop a computational strategy for phenotype prediction (FIG. 1C).

FIG. 1 is a schematic illustration of a study methodology combining multi-omic data analysis with computational strategies for phenotype classification in the context of rheumatoid arthritis (RA) research. Each of FIGS. 1A-1C explain different aspects of the study. FIG. 1A shows icons representing three groups of study participants: ACPA− RA patients, ACPA+ RA patients, and healthy controls, each group consisting of 40 individuals. A blood sample is collected from each participant for deep multi-omic profiling, which includes proteomics, metabolomics, and autoantibodies examination.

FIG. 1B depicts multi-omics analysis, involving comparison between different groups: ACPA− RA vs. ACPA+ RA, ACPA+ RA vs. Controls, and ACPA− RA vs. Controls. The process includes statistical analysis for differential abundance, functional enrichment, and correlations; set comparisons; and network-based phenotype associations.

FIG. 1C depicts computational strategies for phenotype classification; in particular, a computational approach that employs cross-validation (1-fold to 5-fold) and involves sub steps:

1. Network inference using ElasticNet, leading to sub-network mining.
2. Network propagation using a Random walk with further 1-fold to 5-fold validated sub-network mining.
3. The resulting features are then put through feature selection, presented as a heatmap matrix.
4. These selected features are applied to the Random forests algorithm.
5. The performance of the classification is then evaluated.

The elements depicted include detailed representations of molecular structures, network graphs, and computational icons such as heatmaps and the Random forests logo, indicating various data processing and analysis techniques.

FIGS. 1A-1C show an overall methodology for phenotype classification by integrating different data types (proteomics, metabolomics, etc.) and analytical approaches (network analysis, statistical models, etc.) to study the complex nature of rheumatoid arthritis and identify distinct subtypes or patterns based on biomarkers.

In some aspects, a method for classifying rheumatoid arthritis (RA) phenotypes using a combination of multi-omic data analysis and computational strategies may be provided. The method may include collecting blood samples from subjects, including ACPA− RA patients, ACPA+ RA patients, and healthy controls, for deep multi-omic profiling. This profiling may include the examination of proteomics, metabolomics, and autoantibodies to generate biomolecular data for each subject.

The method may include a multi-omics analysis step, where the biomolecular data from the different groups are compared to identify differential abundance, functional enrichment, and correlations among the biomolecules. This analysis aids in distinguishing between the ACPA− RA and ACPA+ RA phenotypes and differentiating these from the control group. The comparison may include statistical analyses, set comparisons, and network-based phenotype associations to elucidate the complex biomolecular interactions underlying RA.

Subsequently, the method may include a computational strategy for phenotype classification. This may include one or more machine learning algorithms to analyze the multi-omic data. For example, this strategy may include network inference through ElasticNet for sub-network mining, network propagation via Random walk for further sub-network mining, and feature selection presented through a heatmap matrix. The selected features may then be applied to a Random forests algorithm for phenotype classification. The performance of the classification model is evaluated through cross-validation techniques, ranging from 1-fold to 5-fold validation.

In general, ElasticNet is a tool used in statistics and machine learning that helps find the best way to predict or classify information based on past data. In particular, ElasticNet helps to determine the most useful factors and combining them in a manner so as to make a prediction as accurate as possible. Generally, a random walk is a process that describes a path consisting of a series of random steps, such as being blindfolded and taking steps in random directions. The path is unpredictable and varies each time. In the context of data analysis, a random walk helps in exploring data by moving through it step by step randomly, which can reveal hidden patterns or connections between different pieces of information. Generally, a heatmap matrix can be visualized to show different values represented by colors. A heatmap matrix can show which factors are more important or which conditions are more common by using a color scale, making it easier to see patterns or trends at a glance. In general, a random forest is a method that combines the predictions from many different trees (decision trees) to make a more accurate overall prediction.

Together, these tools help in analyzing complex data by identifying the most relevant information (ElasticNet), exploring data in an unstructured way to find patterns (random walk), visually highlighting important findings (heatmap matrix), and making accurate predictions by combining multiple analyses (random forests). In the context of classifying rheumatoid arthritis phenotypes, these methods work together to sift through vast amounts of biological data, identify key biomarkers, and accurately classify patients based on their specific disease characteristics, leading to better diagnosis and treatment options.

The present techniques may integrate various data types and analytical approaches to study the complex nature of rheumatoid arthritis. By employing deep multi-omic profiling and advanced computational strategies, the method aims to identify distinct RA subtypes or patterns based on biomarkers, thereby facilitating more precise diagnosis and treatment strategies for RA patients.

To implement the techniques for making predictions, diagnostics, and treatments of rheumatoid arthritis (RA) phenotypes through multi-omic data analysis and computational strategies, a sophisticated computer system may be required. This system may need to be capable of handling large volumes of complex biological data and executing advanced computational algorithms. Such a computer system may include hardware components including: (1) a CPU with multiple cores for processing the complex algorithms involved in multi-omic data analysis and machine learning; (2) a GPU for training deep learning models and performing high-throughput computations required in network analysis; (3) RAM to hold large datasets and intermediate computations in memory, allowing for faster access and processing of data; and (4) magnetic or solid-state drives (SSDs) for storing and accessing large multi-omic datasets, software tools, and the results of analyses. The computing systems may include networking hardware to facilitate the transfer of large data sets between the computer system and external databases or cloud services.

The computing system may include the following software components: (1) a stable and secure operating system that can efficiently manage the system's resources while providing support for the necessary data analysis and machine learning software; (2) software tools specialized for bioinformatics and multi-omic data analysis, that support data preprocessing, statistical analysis, and the visualization of complex datasets, such as heatmaps and network diagrams; (3) software frameworks that support a wide range of machine learning algorithms, including ElasticNet, random forests, and network propagation models, and which allow for the customization and optimization of algorithms for specific tasks in RA phenotype classification; (4) database management software to organize and manage the vast amounts of multi-omic data, computational models, and analysis results, a robust database management system. For particularly demanding computational tasks, access to a high performance computing (HPC) environment or cloud computing resources may be used to provide additional computational power. This may include specialized software for distributing computations across multiple processors or nodes.

### RA Subgroup-Specific Characteristics in Multi-Omic Profiles

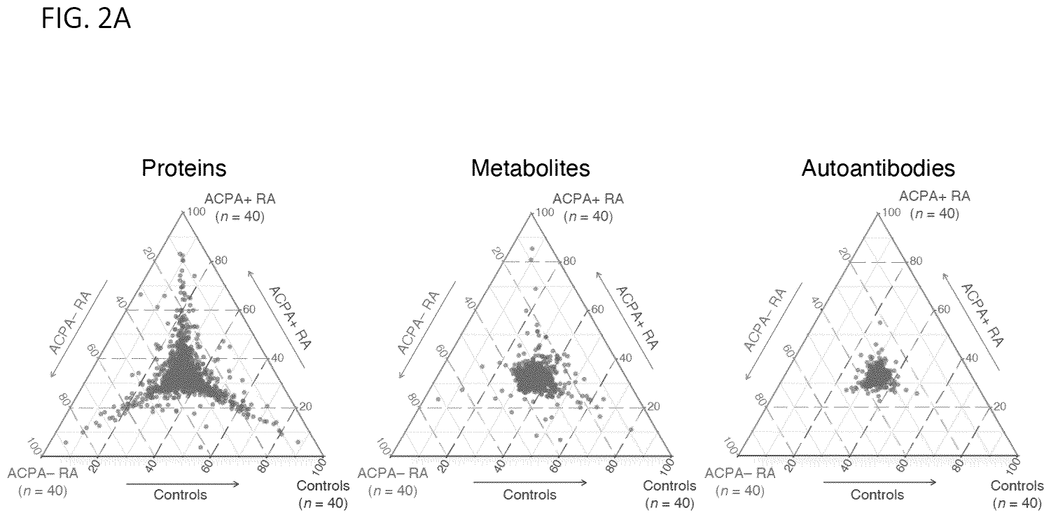

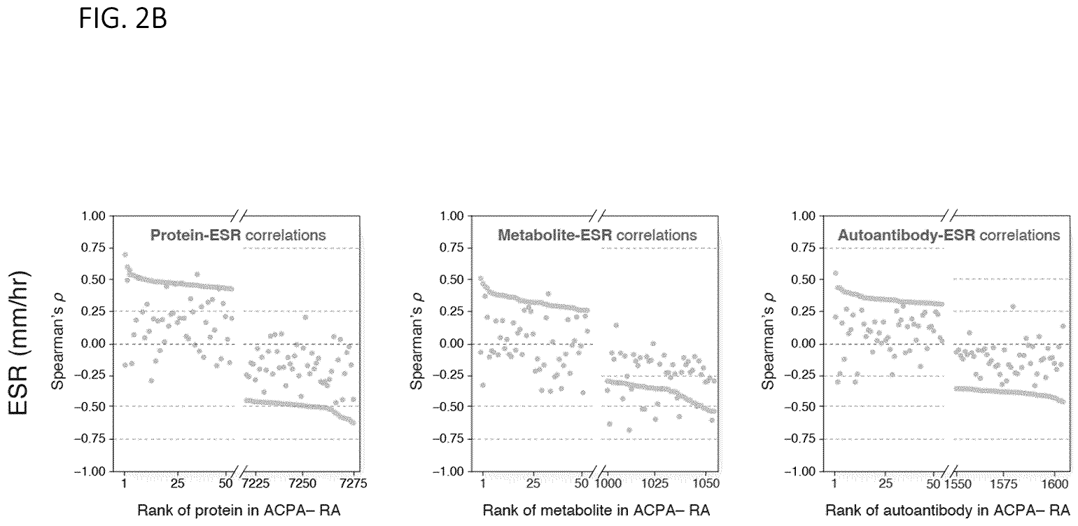

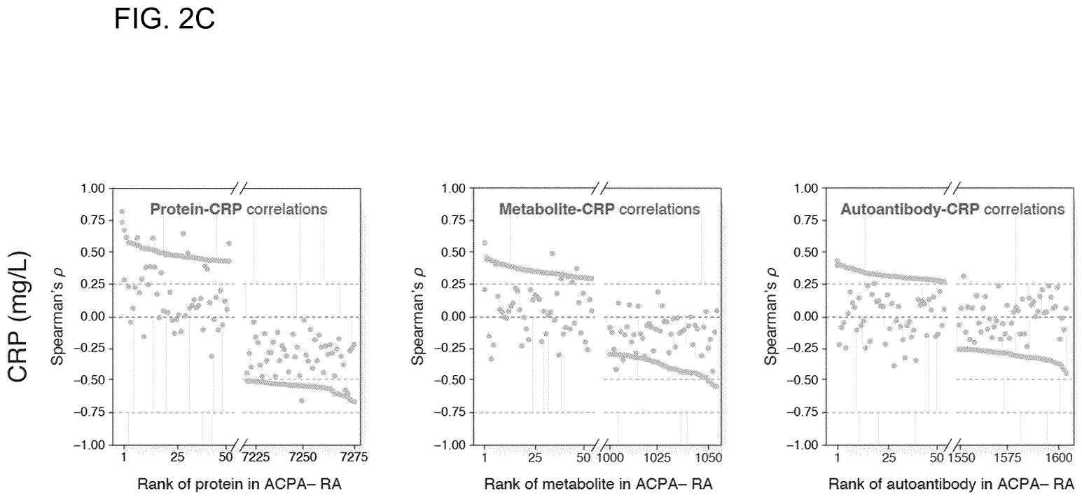

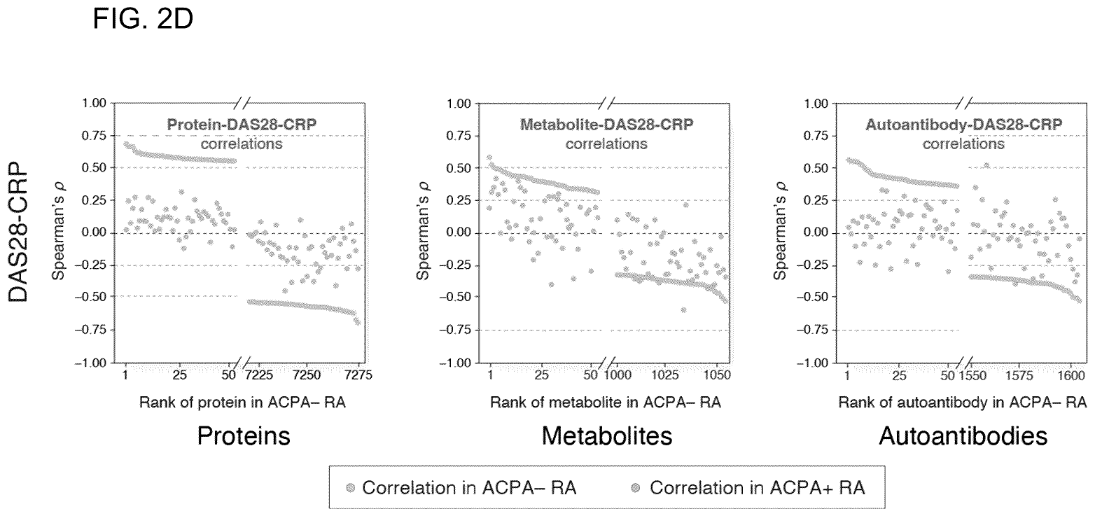

The heterogeneity of omic features was evaluated across the three study groups, i.e., ACPA− RA, ACPA+ RA, and controls. As shown in FIG. 2A, proteins exhibited the most features that were specific to each study group. Similarly, the metabolite profiles exhibited notable differences among these groups, albeit to a substantially lesser extent than the protein profiles. In contrast, most autoantibodies were observed to maintain broadly similar mean abundances across all three groups.

Building on the observed group-specific omic features, the differences in associations, particularly correlations between omic features and clinical characteristics within the ACPA− RA and ACPA+ RA subgroups, were investigated next. The rank plots shown in FIG. 2B illustrate the correlations of omic features with three parameters: the blood acute phase inflammatory markers ESR and CRP, and DAS28-CRP (a quantitative measure of disease activity). The distinct separation between the gold points (representing correlations in ACPA− RA) and the gray points (representing correlations in ACPA+ RA) indicate significant disparities in the top positive and top negative correlations (FIGS. 2B-2D). The differences in correlations between circulating biomolecules and these particular clinical characteristics may imply underlying variations in inflammatory responses between the two RA subgroups. Select examples that merit further discussion are elaborated below:

In the ACPA− RA subgroup, the analysis found Matrix Metallopeptidase 19 (MMP19) protein as having the most positive correlation with ESR (p=0.67 and P=2.24×10⁻⁶). This marked correlation did not extend to the ACPA+ RA subgroup, wherein the correlation between MMP19 and ESR was not significant (ρ=−0.17 and P=0.30). Interestingly, MMP19 also correlated positively with CRP (ρ=0.49 and P=1.47×10⁻³) in ACPA− RA, but again, this association was absent in ACPA+ RA (p=0.03 and P=0.87). While the specific role of MMP19 in RA is yet to be fully elucidated, its identification as an autoantigen in the inflamed synovium of an RA patient suggests its potential involvement in disease (27).

Within the ACPA− RA subgroup, the analysis revealed 5 metabolites (1-palmitoyl-2-stearoyl-GPC (16:0/18:0), 5-(galactosylhydroxy)-lysine, N-stearoyl-sphingosine (d18:1/18:0), 4-hydroxyphenylpyruvate, and quinolinate) that had significant positive correlations with ESR, each with a Spearman's p exceeding 0.4 and a corresponding P-value below 0.05. In contrast, within the ACPA+ RA subgroup, these metabolites either exhibited negative correlations (ρ<0 and P<0.05) or showed no significant correlation (P≥0.05). Additionally, within the ACPA− RA subgroup, 14 metabolites, which demonstrated significant negative correlations (ρ<−0.4 and P<0.05) with ESR were identified including biliverdin, bilirubin (Z,Z), and a bilirubin degradation product (C17H20N2O5). In the ACPA+ RA subgroup, however, these correlations were either positive (ρ>0 and P<0.05) or non-significant (P≥0.05).

Previous studies in RA have reported negative correlations between bilirubin-derived metabolites and disease activity in RA (28-30). Considering these reports, the correlations between these metabolites and clinical characteristics in the present datasets were investigated to identify potential differences between the ACPA− and ACPA+ RA subgroups. The analysis confirmed that 2 bilirubin-derived metabolites (i.e., bilirubin degradation product [C16H18N2O5], bilirubin [E,Z or Z,E]) exhibited negative correlations with DAS28-CRP (ρ<−0.4 and P<0.05) in both the ACPA− and ACPA+ RA subgroups. However, disparate correlations between bilirubin-derived metabolites and the acute phase inflammatory markers (ESR and CRP) were identified in ACPA− and ACPA+ RA. For instance, in the ACPA− RA subgroup, biliverdin and bilirubin (Z,Z) were both negatively correlated with ESR (biliverdin: ρ=−0.52 and P=5.11×10−4; bilirubin (Z,Z): ρ=−0.48 and P=1.73×10−3) and CRP (biliverdin: ρ=−0.45 and P=3.28×10−3; bilirubin (Z,Z): ρ=−0.43 and P=5.19×10−3). Conversely, within the ACPA+ RA subgroup, neither biliverdin nor bilirubin (Z,Z) showed significant correlations with ESR (biliverdin: ρ=−0.29 and P=0.08; bilirubin (Z,Z): ρ=−0.13 and P=0.44) and CRP (biliverdin: ρ=−0.24 and P=0.15; bilirubin (Z,Z): ρ=−0.02, P=0.89).

Plasma Proteomic Profiling Reveals Proteins that Differ Between ACPA− and ACPA+ RA

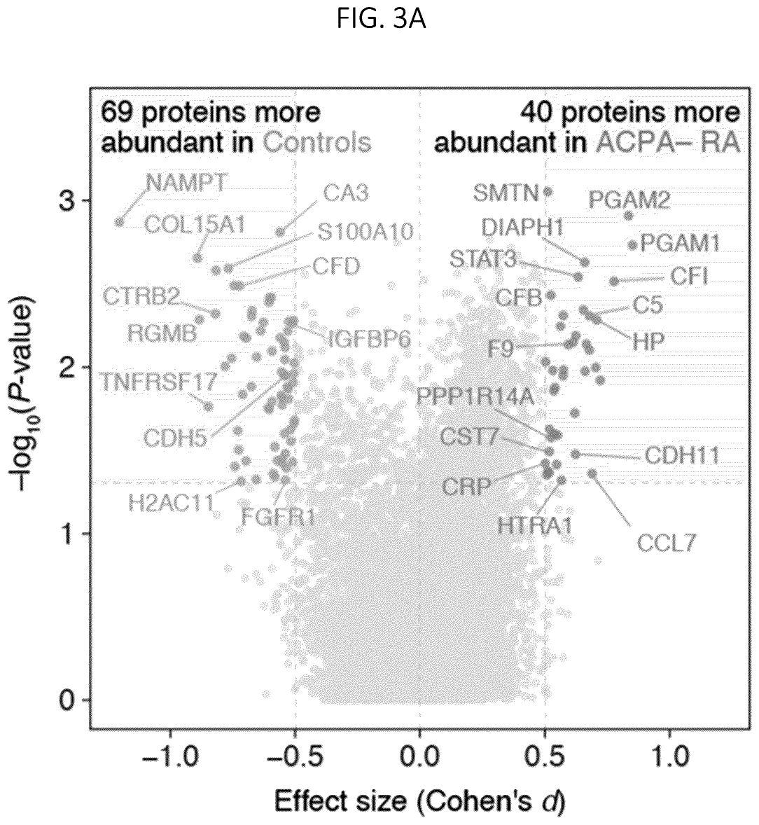

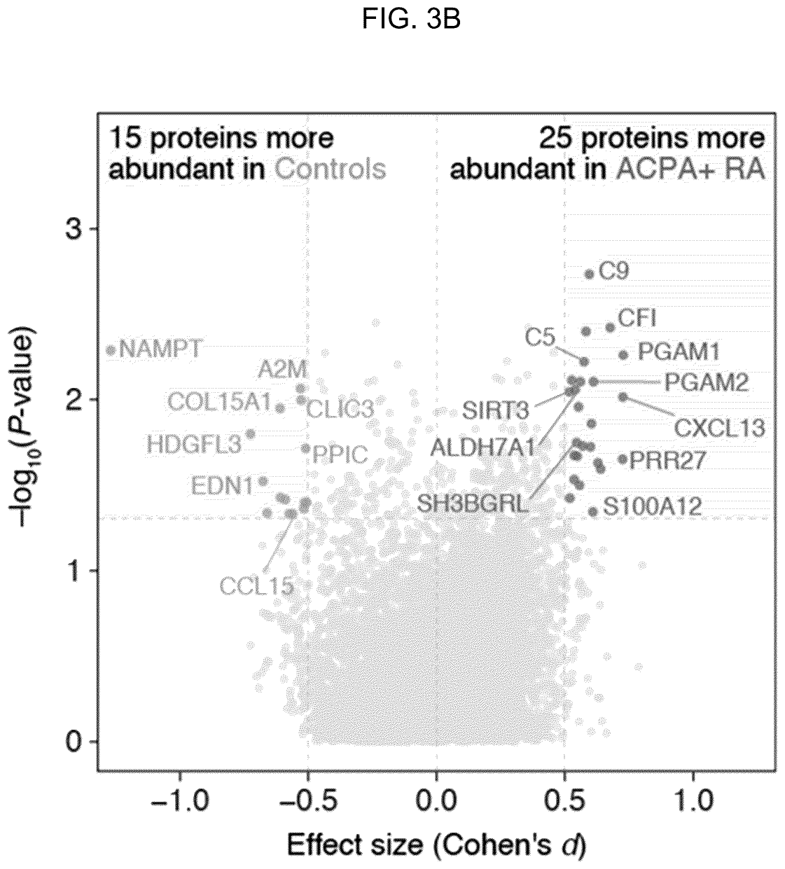

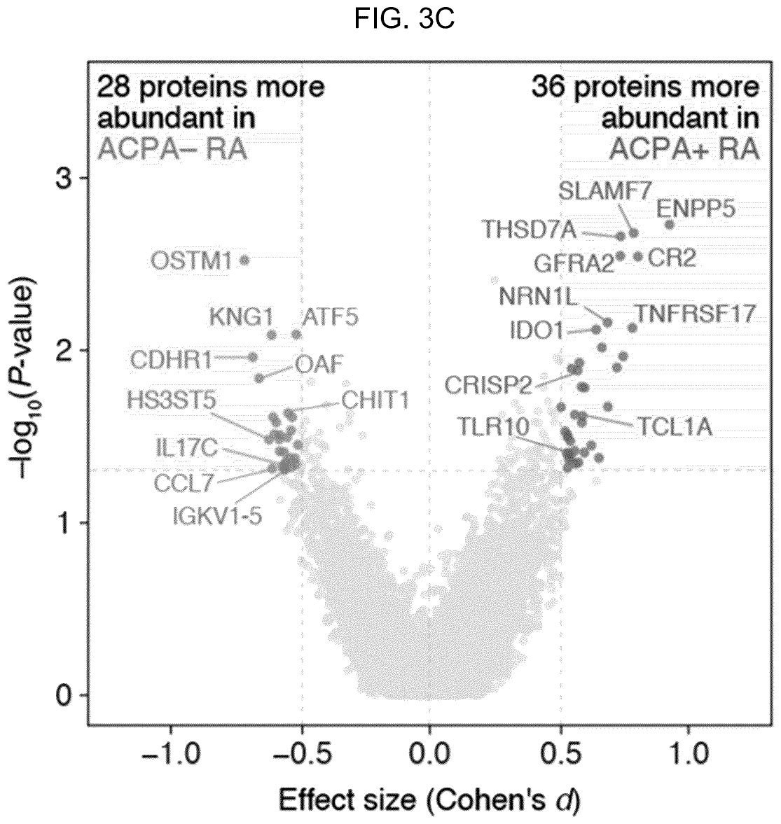

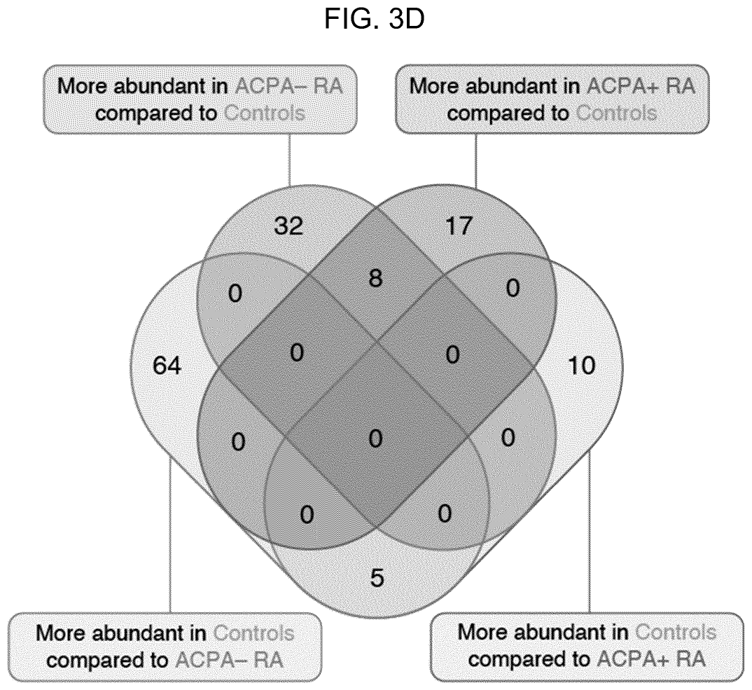

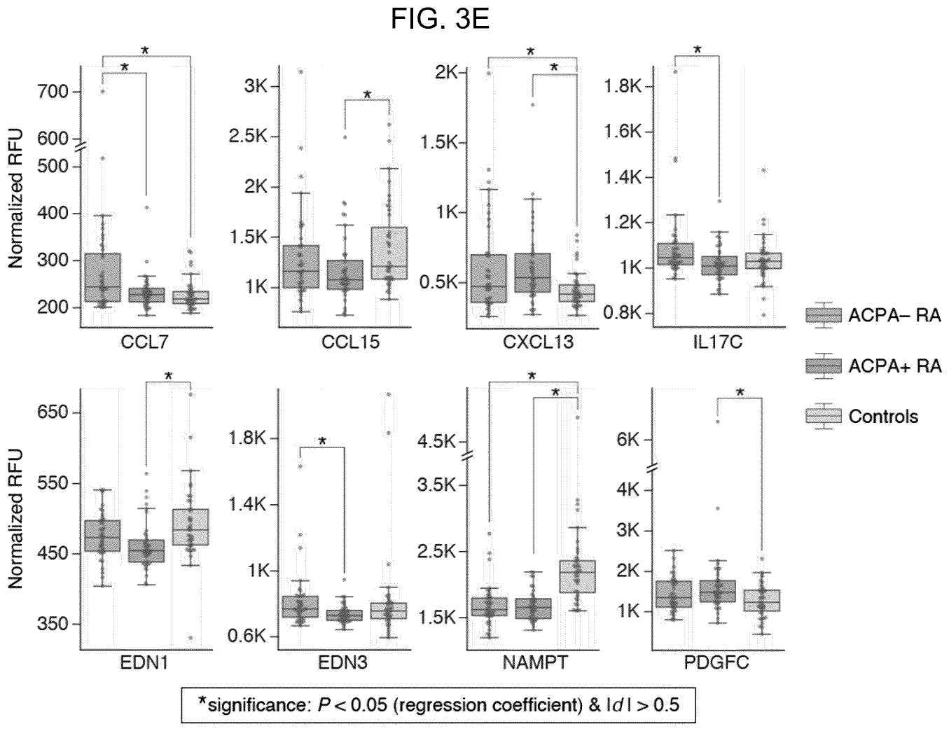

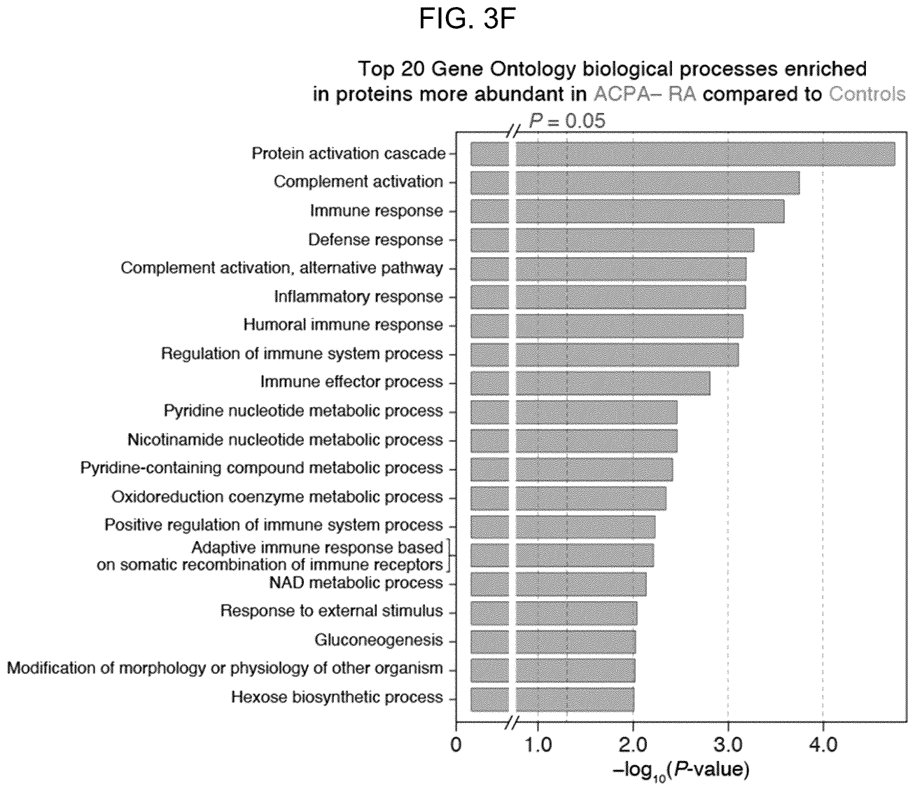

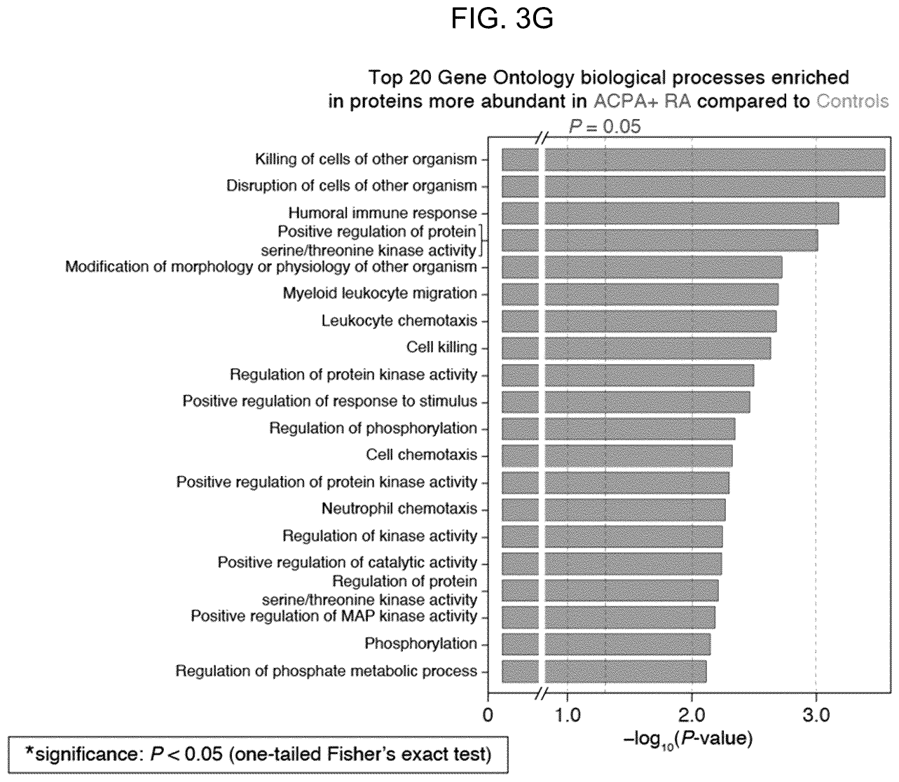

The identification of distinct correlations between clinical characteristics and proteins in ACPA− RA and ACPA+ RA motivated further investigations on the differences in the abundance of individual plasma proteins between study groups. For this, a thorough analysis in search of group-associated proteins was conducted, whereby differentially abundant proteins were selected while controlling for potential confounding factors (i.e., sex, age, BMI, smoking history, and use of prednisone, bDMARDs, csDMARDs) (Materials and Methods). This analysis was structured into three pair-wise group comparisons: ACPA− RA vs. controls, ACPA+ RA vs. controls, and ACPA+ RA vs. ACPA− RA. Among 7,273 proteins, 40 proteins with higher abundance and 69 with lower abundance in ACPA− RA compared to controls were identified (FIGS. 3A-3C). In contrast, ACPA+ RA showed 25 proteins with higher abundance and 15 with lower abundance than controls. Additionally, when comparing ACPA+ RA directly with ACPA− RA, 36 proteins were found to be more abundant in ACPA+ RA, while 28 proteins were more abundant in ACPA− RA.

Among the 32 proteins identified to be exclusively more abundant in ACPA− RA (but not in ACPA+ RA) compared to controls (FIG. 3D), 10 proteins (CCL7, CFB, CFHR5, CRP, CST7, FGL1, HP, IL1RN, KYNU, and STAT3) belonging to the Gene Ontology (GO) term “Immune response” (GO: 0006955) were identified. Conversely, a different set of “Immune response” proteins (C9, MSRB1, RAC2, and S100A2) were found among a total of 17 proteins that were more abundant in only ACPA+ RA (but not in ACPA− RA) compared to controls (FIG. 3D). Additionally, the study identified 3 “Immune response” proteins (CXCL13, C5, and CFI) among 8 which were commonly found in higher abundance in both ACPA− and ACPA+ RA subgroups relative to controls (FIG. 3D).

The observation of differences in the abundance of immune proteins between ACPA− and ACPA+ RA subgroups led to specific examinations of levels of different cytokines utilizing the cytokine registry from ImmPort (31). As shown in FIG. 3E, notable differences were observed, for example: The monocyte chemoattractant protein 3 (CCL7), which has been reported to be highly expressed in RA synovium and correlated with anti-cyclic citrullinated peptide (anti-CCP) antibodies (32), was significantly more abundant in ACPA− RA compared to ACPA+ RA and controls. However, plasma CCL7 levels in ACPA+ RA patients did not differ significantly from those in controls, aligning with findings from Rump et al. (32). Conversely, platelet-derived growth factor C (PDGFC) showed higher abundance in ACPA+ RA than in controls. Meanwhile, B cell-attracting chemokine 1 (CXCL13) was more abundant in both RA subgroups compared to controls, possibly pointing to a shared B cell recruitment pathway. Additionally, C-C motif chemokine 15 (CCL15) and endothelin-1 (EDN1) were more abundant in controls than in ACPA+ RA, while pre-B cell-enhancing factor (NAMPT) was higher in controls compared to both RA subgroups. Moreover, interleukin 17C (IL17C) and endothelin-3 (EDN3) were identified as being more abundant in ACPA− RA compared to ACPA+ RA. Overall, these findings suggest that ACPA− and ACPA+ RA patients may be associated with different mechanisms of immune cell recruitment in blood (7, 33).

To gain a comprehensive understanding of functional differences (as reflected through blood proteins) between ACPA− and ACPA+ RA, enriched GO biological processes were explored in DAVID (34). The focus was on proteins that were more abundant in ACPA− RA (40 proteins) and ACPA+ RA (25 proteins) compared to controls, which were found to be enriched in 63 and 60 biological processes, respectively. Notably, immune-related GO terms, such as “Humoral immune response”, “Defense response”, and “Adaptive immune response based on somatic recombination”, were commonly enriched in both RA subgroups (FIGS. 3F-3G).

While both RA subgroups shared immune-related enrichments, a striking distinction emerged in the enrichment of metabolism-associated GO terms—even after adjusting for factors that significantly influence human metabolism (BMI, prednisone use). Specifically, ACPA− RA had 19 of 63 GO terms related to metabolic processes, whereas ACPA+ RA had 7 of 60. Notably, there were no overlapping (enriched) metabolic processes between the two RA subgroups: GO terms related to carbohydrate metabolism (e.g., “Glycolytic process”, “Pyruvate metabolic process”, “Carbohydrate catabolic process”) were uniquely enriched in ACPA− RA; whereas ACPA+ RA showed unique enrichment in processes related to phosphate/phosphorus metabolism (e.g., “Phosphorus metabolic process”, “Regulation of phosphate metabolic process”).

Plasma Metabolites are Associated with ACPA Status and Age Group

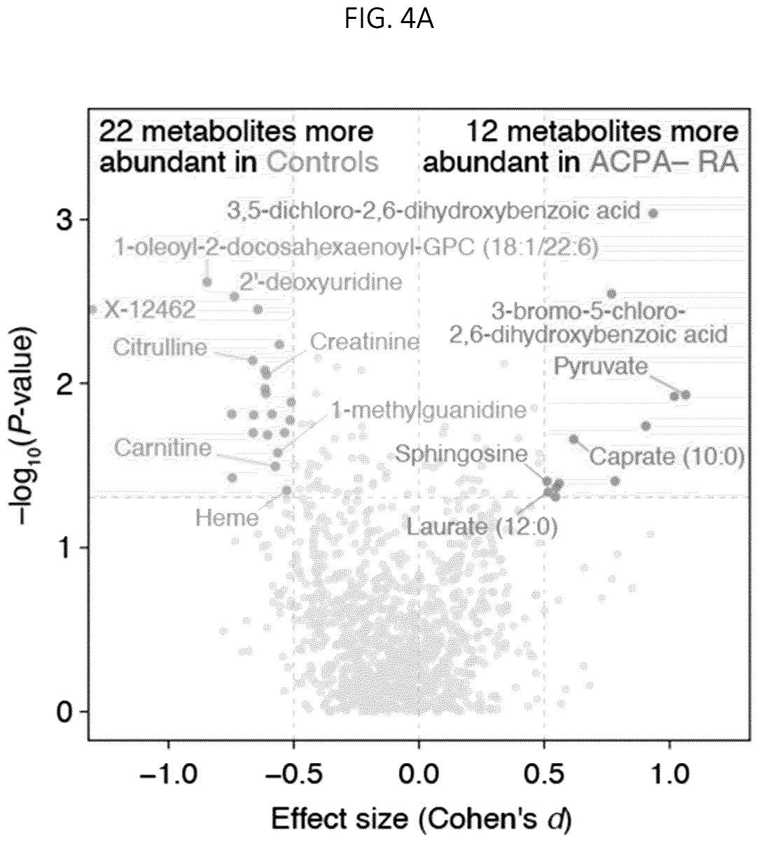

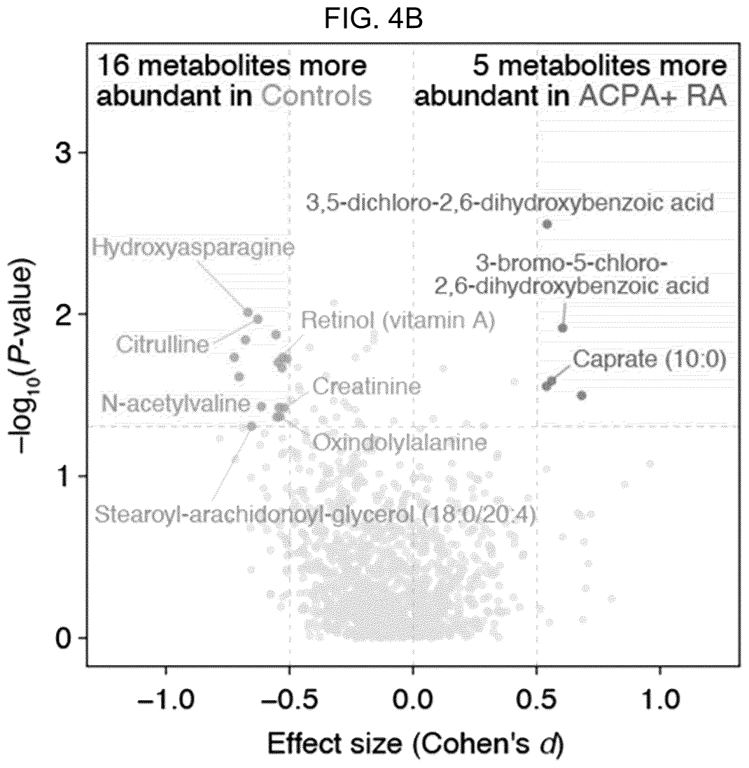

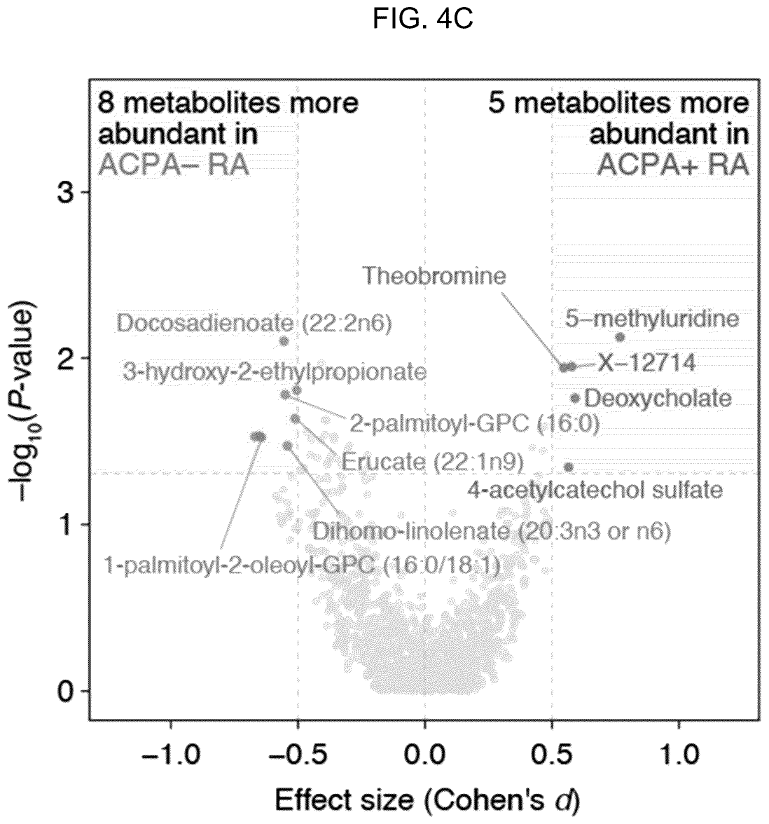

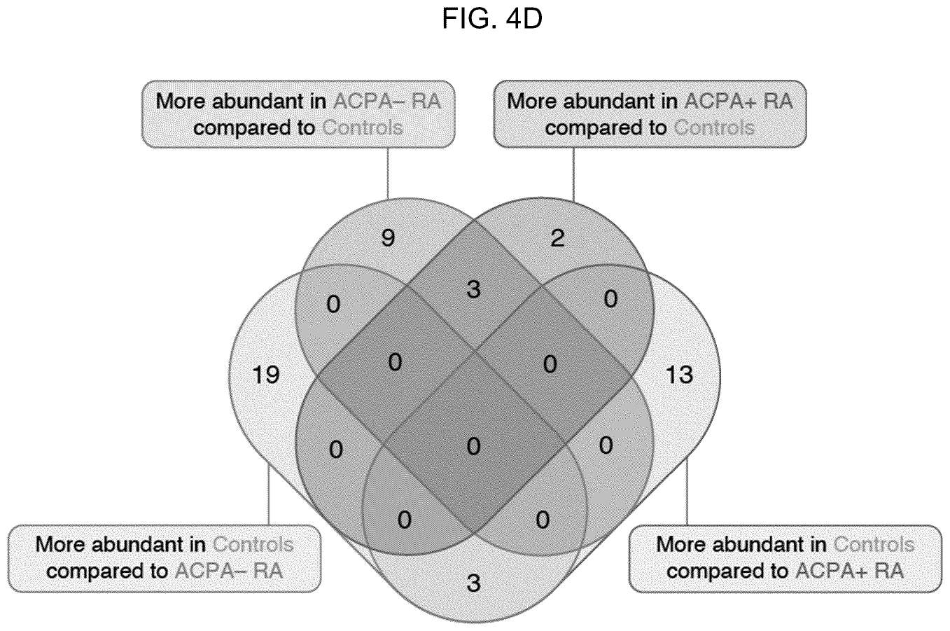

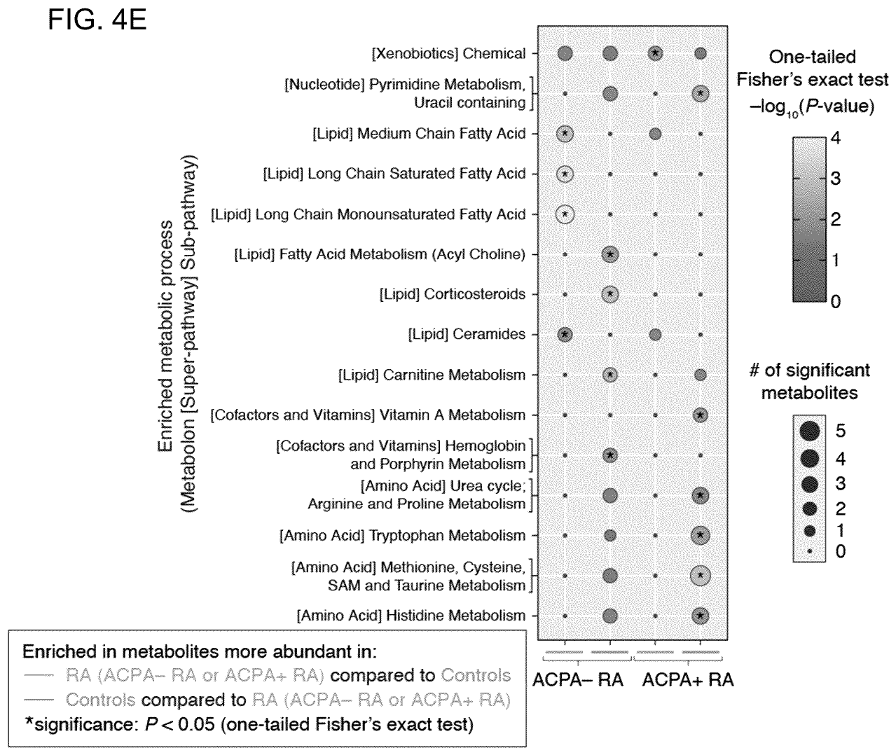

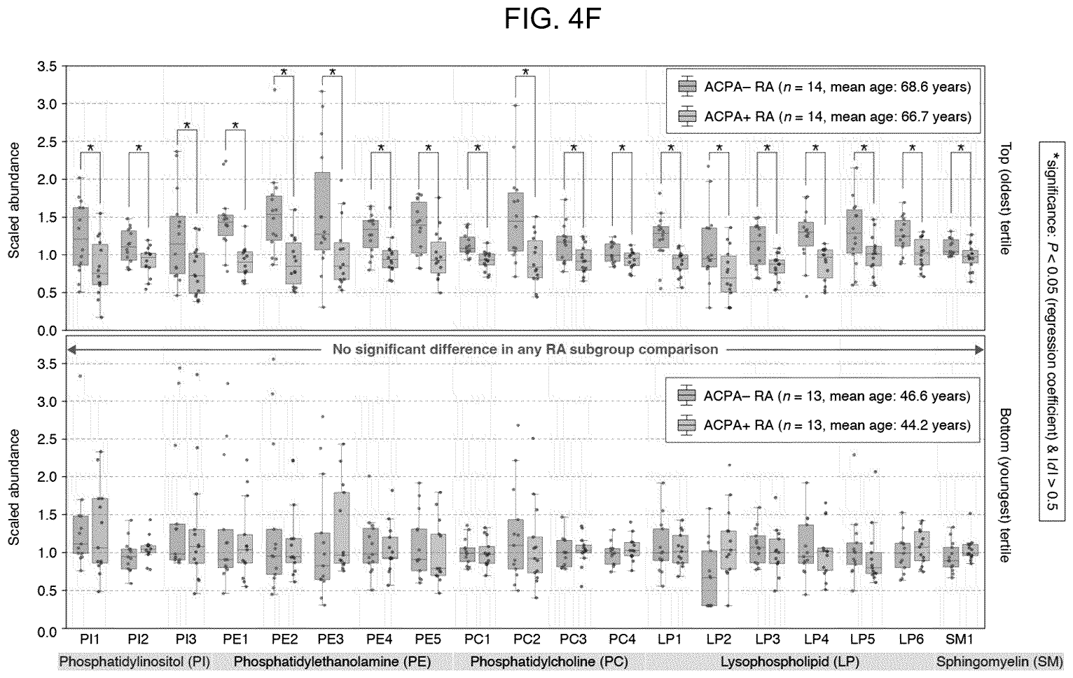

Next, plasma metabolites were investigated to uncover new insights into metabolism that may not be accessible through proteomics-based analyses. In the analysis of 1,061 metabolites, 12 metabolites were found to be significantly more abundant in ACPA− RA and 22 metabolites were less abundant than in controls (FIGS. 4A-4C). In ACPA+ RA, there were 5 metabolites with higher abundance and 16 with lower abundance relative to controls. Additionally, 5 metabolites were found to be more abundant in ACPA+ RA than in ACPA− RA, while 8 metabolites were more abundant in ACPA− RA than in ACPA+ RA.

Nine metabolites (including pyruvate, laurate (12:0), and sphingosine) were identified as being uniquely more abundant in ACPA− RA (but not in ACPA+ RA) compared to controls; and 2 metabolites (i.e., branched-chain/straight-chain/cyclopropyl 10:1 fatty acid, X-23276) were uniquely more abundant in ACPA+ RA (but not in ACPA− RA) compared to controls (FIG. 4D). The differential level of pyruvate found exclusively in ACPA− RA aligns with the proteomics results, wherein proteins were enriched in glycolytic and pyruvate metabolic processes for ACPA− RA but not for ACPA+ RA. Of note, other metabolites of the “Glycolysis, gluconeogenesis, and pyruvate metabolism” GO term, such as glucose, glycerate, lactate, 1,5-anhydroglucitol, and 3-phosphoglycerate, did not show a similar association with either ACPA− RA or ACPA+ RA.

To comprehensively elucidate the metabolic processes implicated in ACPA− and ACPA+ RA, a statistical enrichment analysis was conducted on metabolites more abundant in each RA subgroup compared to controls. Fifteen metabolic subpathways were discovered to be enriched across two pair-wise group comparisons: ACPA− RA vs. controls or ACPA+ RA vs. controls (FIG. 4E). Strikingly, there were no subpathways that were commonly enriched in both ACPA− and ACPA+ RA, indicating their distinct metabolomic profiles: 8 subpathways (e.g., “Medium chain fatty acid”, “Long chain fatty acid”) were exclusively enriched in ACPA− RA, and 7 subpathways (e.g., “Pyrimidine metabolism, uracil containing”, “Urea cycle, arginine and proline metabolism”) were unique to ACPA+ RA. Particularly noteworthy is the enrichment of subpathways involved in lipid metabolism for only ACPA− RA, such as “Medium chain fatty acid”, “Long chain saturated fatty acid”, “Long chain monounsaturated fatty acid”, and “Ceramides.”

Recognizing the significant influence of age on blood lipid composition (35, 36), and the emerging evidence linking plasma lipid changes with RA (37, 38), complex lipids that could be sensitive to both age and ACPA status in RA patients were investigated. For this, each study group (ACPA− RA, ACPA+ RA, and controls) was further divided into three age groups: youngest (bottom tertile, n=13), intermediate (middle tertile, n=13), and oldest (top tertile, n=14). Then, complex lipids differentiating ACPA− and ACPA+ RA were searched for each age-stratified tertile. As a result, 19 lipids that were differentially abundant were identified; interestingly, all 19 were significantly more abundant in the oldest age group (average age of 67 years) of ACPA− RA patients compared to their ACPA+ RA counterparts (FIG. 4F). These complex lipids were categorized into the following subclasses: phosphatidylinositol, phosphatidylethanolamine, phosphatidylcholine, lysophospholipid, and sphingomyelin. Strikingly, no such differences were seen in the youngest age group (average age of 45 years), and none of these 19 lipids showed a significant association with either ACPA− RA or ACPA+ RA when compared to controls in any age group. Though the mechanisms behind these age-related complex lipid variations are yet unclear, these results suggest a unique lipidomic signature associated with aging—particularly for elderly patients with ACPA− RA.

### Autoantibody Signatures Differentiating ACPA− RA and ACPA+ RA

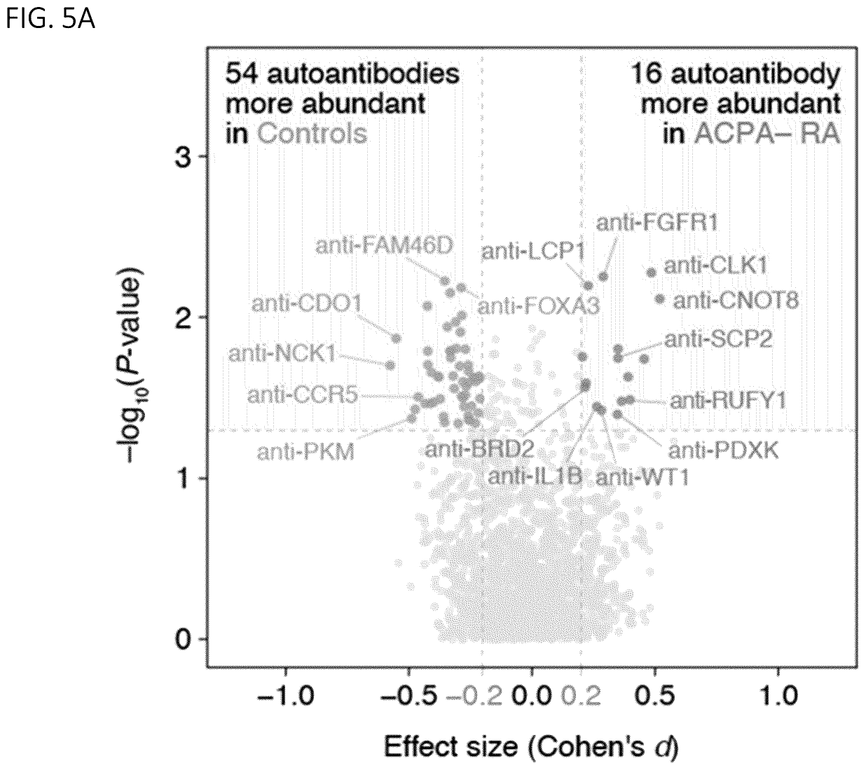

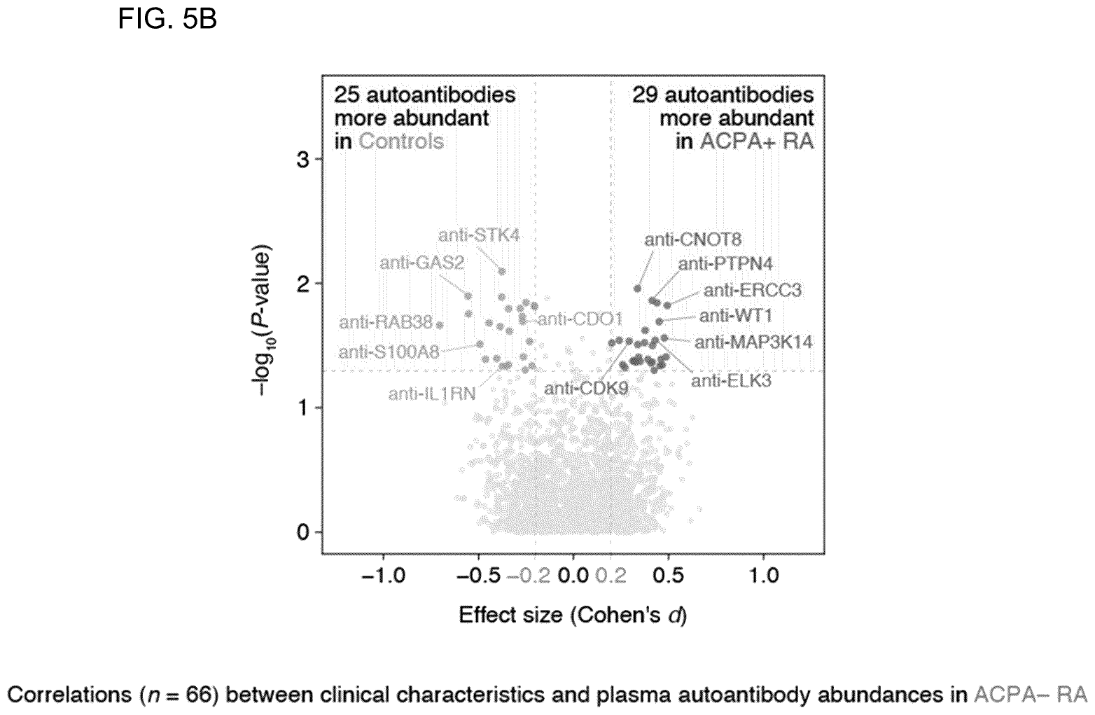

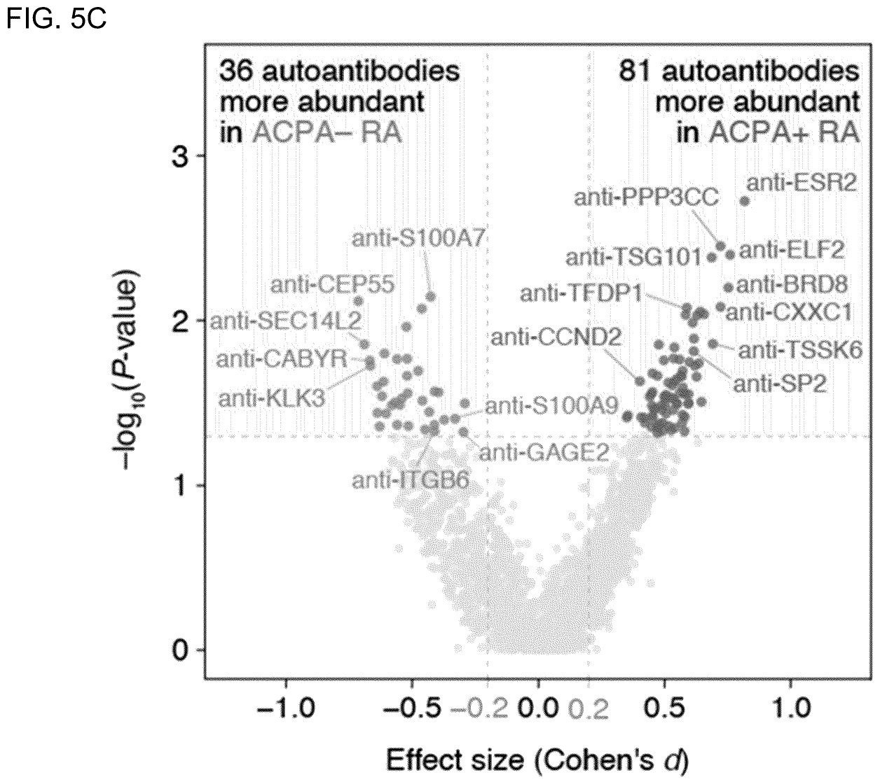

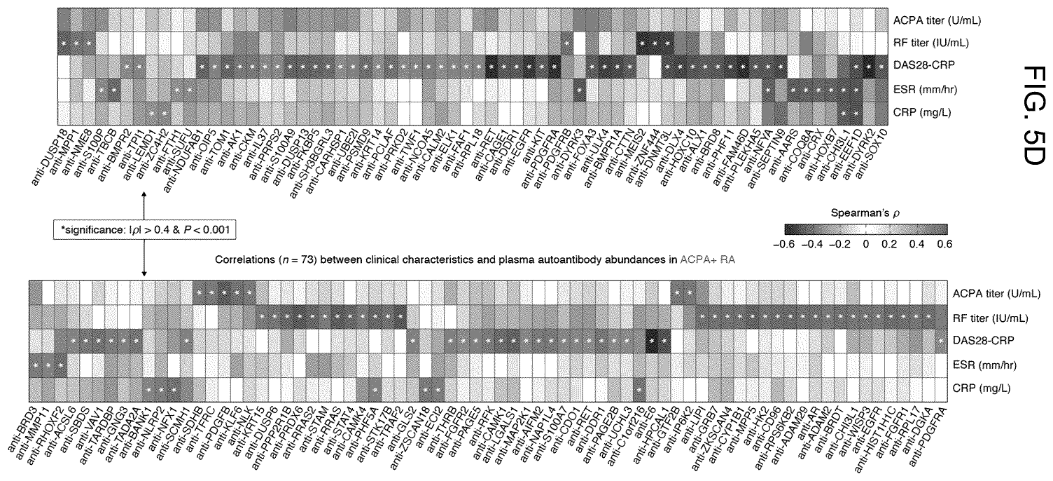

1,610 autoantibodies in plasma from our three study groups were analyzed to identify those significantly associated with ACPA− or ACPA+ RA. In ACPA− RA, 16 autoantibodies were found to be more abundant and 54 to be less abundant than in control subjects (FIGS. 5A-5C). Conversely, in ACPA+ RA, 29 autoantibodies were more abundant, while 25 were less abundant compared to controls. Notably, 3 autoantibodies (anti-WT1, anti-CLK1, and anti-CNOT8) showed increased abundance in both RA subgroups. A direct comparison between the ACPA− and ACPA+ RA subgroups revealed more pronounced differences: 81 autoantibodies were significantly more abundant in ACPA+ RA, and 36 in ACPA− RA.

Since autoantibodies are known to play a critical role in chronic inflammation (39, 40), the distinct autoantibody profiles between ACPA− and ACPA+ RA led to further investigations (as a post hoc analysis) on the relationship between plasma autoantibodies and the following clinical characteristics: ESR and CRP, rheumatoid factor (RF) and ACPA titers, and DAS28-CRP. Sixty-six significant correlations (i.e., |Spearman's ρ|>0.4 and P<0.01) were between autoantibodies and clinical characteristics in ACPA− RA (FIG. 5D), while 73 correlations were found in ACPA+ RA (FIG. 5D). Notably, correlations involving only 2 autoantibodies (anti-PDGFRA and anti-CHI3L1) were common to both RA subgroups: anti-PDGFRA negatively correlated with DAS28-CRP in both RA subgroups, while anti-CHI3L1 negatively correlated with RF titer in ACPA+ RA and with ESR and CRP in ACPA− RA.

### Multi-Omics Analysis Reveals Inverted Biomolecular Correlations Between RA Subgroups

The exploration into the intricate multi-omic landscape of ACPA− and ACPA+ RA has not only characterized unique biomolecules associated with each RA subgroup, but also uncovered subgroup-specific biological processes, metabolic pathways, and even the directionality of pair-wise feature correlations. However, translating these findings into clinical advancements remains challenging due to the need to convert statistical associations into actionable predictions. This is particularly important in diagnosing ACPA− RA, where specific tests are lacking. Leveraging multi-omic data to develop predictive diagnostics for ACPA− RA could address this. Thus, the effectiveness of blood-derived proteins, metabolites, and autoantibodies in distinguishing ACPA− RA from controls was investigated next. Additionally, to broaden the utility of the potential diagnostic tools, the effectiveness of these multi-omic features to differentiate ACPA+ RA from controls, and also distinguish between the RA subgroups, was evaluated.

Identifying a clinically meaningful subset of features from over 9,000 biomolecules requires a robust machine learning framework that holistically integrates complex interactions with phenotypic data (i.e., study group). As an initial step, all measured features were integrated into a global multi-omic network. This expansive network, consisting of 9,949 nodes and 322,491 edges, was constructed from an iterative elastic net penalized regression analysis that incorporates each feature in conjunction with clinical and demographic data (see Materials and Methods for details). Subsequently, to streamline the feature selection process, a network diffusion technique was applied, specifically a random walk with restart algorithm, that navigates the network's topological structure to prioritize features closely linked to the phenotype node. This approach led to a refined subnetwork, (FIG. 6A), which focuses on a subset of 30 nodes connected by 66 edges that were most closely associated with the phenotype. Encouragingly, this targeted network corroborated the prior single-omic results, with 11 of the 30 features exhibiting significant differential abundance between ACPA− RA and controls or ACPA+ RA and controls (FIG. 6B). Notably, the network pinpointed features associated with glucose metabolism (pyruvate and lactate) as predictive features in the penalized regression models, thereby elucidating their (as well as other features') potential utility in subgroup classification.

Finally, to assess the efficacy of the network-based approach in phenotype classification, the effectiveness of the methodology—comprising network inference and subsequent feature prioritization and selection—to differentiate between ACPA− RA, ACPA+ RA, and control groups was investigated. Implementing a 5-fold cross-validation scheme, the strategy utilizes network inference, network diffusion, and the top-selected features to train a random forest classifier (for RA subgroup classification) in each K-fold dataset. The network-based machine learning strategy differentiated ACPA− RA patients and controls with an area under the receiver operating characteristic curve (AUC) of 0.95; distinguished ACPA+ RA patients from controls with an AUC of 0.93; and separated RA subgroups (ACPA− and ACPA+ RA together) from controls with an AUC of 0.91 (Table 2). Additionally, this multi-omic-based strategy generally achieved better AUCs compared to strategies trained on single omic data types (Table 2). These findings highlight the integrative machine learning strategy as a promising computational biomarker discovery platform for RA diagnostics, especially in patients who test negative for ACPA.

Building on the strategy's high proficiency in distinguishing RA subgroups from controls, further evaluations were performed on its ability to distinguish between ACPA− RA and ACPA+ RA. Although the clinical relevance of this distinction is not immediately evident as ACPA status is already known, an exploration was nevertheless conducted on whether the differential abundance of plasma omics measurements could support a classification model for discerning these two specific classes. Notably, the metabolomics data yielded the highest AUC of 0.71 (Table 2), suggesting that metabolic differences may be most pronounced between the two subgroups; other omic data types and combined omics data achieved AUCs below 0.68 (Table 2). To test the generalizability of these findings, the model's performance was assessed using plasma metabolomics datasets from two independent validation cohorts: 1) the Mayo Clinic Early Arthritis Cohort, comprising 9 ACPA− and 28 ACPA+ RA patients who were newly diagnosed (i.e., disease duration of ≤6 months) and DMARD treatment-naïve; and 2) a cohort from our previously published study by Hur et al. (28) of 28 ACPA− and 103 ACPA+ RA patients with established disease. In these cohorts, our model achieved an AUC of 0.70 and 0.72, respectively, in distinguishing ACPA− RA from ACPA+ RA (Table 3). These results are largely consistent with the performance observed during cross-validation in the training cohort.

TABLE 2
RA subgroup classification performance in 5-fold cross-validation.

| | AUC^{β} | Accuracy | Sensitivity | Specificity | PPV^{γ} | NPV^{δ} |
|---|---|---|---|---|---|---|
| Classification task^{α} | (Mean ± SD) | (Mean ± SD) | (Mean ± SD) | (Mean ± SD) | (Mean ± SD) | (Mean ± SD) |
| ACPA− RA vs. Controls | | | | | | |
| Multi-omics | 0.95 ± 0.05 | 92.5% ± 5.2% | 92.5% ± 6.8% | 92.5% ± 11.2% | 93.5% ± 9.3% | 93.1% ± 6.4% |
| Proteins | 0.90 ± 0.09 | 87.5% ± 8.8% | 90.0% ± 10.5% | 85.0% ± 10.5% | 86.1% ± 10.0% | 89.9% ± 10.5% |
| Metabolites | 0.93 ± 0.09 | 88.8% ± 10.3% | 90.0% ± 10.5% | 87.5% ± 15.3% | 88.9% ± 12.4% | 90.0% ± 9.9% |
| Autoantibodies | 0.59 ± 0.14 | 65.0% ± 7.1% | 67.5% ± 20.9% | 62.5% ± 19.8% | 65.7% ± 10.4% | 70.4% ± 17.5% |
| ACPA+ RA vs. Controls | | | | | | |
| Multi-omics | 0.93 ± 0.07 | 88.8% ± 6.8% | 95.0% ± 6.8% | 82.5% ± 14.3% | 85.5% ± 10.1% | 95.0% ± 6.8% |
| Proteins | 0.75 ± 0.16 | 75.0% ± 11.7% | 67.5% ± 24.4% | 82.5% ± 20.9% | 84.8% ± 17.2% | 74.0% ± 15.3% |
| Metabolites | 0.90 ± 0.09 | 87.5% ± 11.7% | 87.5% ± 8.8% | 87.5% ± 17.7% | 89.0% ± 15.2% | 87.2% ± 9.1% |
| Autoantibodies | 0.62 ± 0.06 | 70.0% ± 5.2% | 67.5% ± 6.8% | 72.5% ± 10.5% | 71.8% ± 8.0% | 69.1% ± 4.8% |
| RA vs. Controls | | | | | | |
| Multi-omics | 0.91 ± 0.06 | 86.7% ± 6.8% | 96.3% ± 3.4% | 67.5% ± 22.7% | 86.3% ± 8.3% | 91.0% ± 8.8% |
| Proteins | 0.82 ± 0.13 | 78.3% ± 10.8% | 91.3% ± 9.5% | 52.5% ± 24.0% | 79.9% ± 9.1% | 75.8% ± 19.2% |
| Metabolites | 0.89 ± 0.09 | 87.5% ± 8.3% | 98.8% ± 2.8% | 65.0% ± 24.0% | 85.7% ± 9.3% | 96.7% ± 7.5% |
| Autoantibodies | 0.51 ± 0.06 | 69.2% ± 2.3% | 95.0% ± 5.2% | 17.5% ± 11.2% | 69.8% ± 2.0% | 70.8% ± 21.0% |
| ACPA− RA vs. ACPA+ RA | | | | | | |
| Multi-omics | 0.68 ± 0.14 | 68.8% ± 14.0% | 65.0% ± 25.6% | 72.5% ± 10.5% | 68.3% ± 13.7% | 70.0% ± 15.4% |
| Proteins | 0.66 ± 0.07 | 67.5% ± 8.1% | 72.5% ± 22.4% | 62.5% ± 12.5% | 65.7% ± 4.8% | 72.9% ± 12.1% |
| Metabolites | 0.71 ± 0.13 | 67.5% ± 11.2% | 75.0% ± 17.7% | 60.0% ± 13.7% | 65.2% ± 9.1% | 72.4% ± 17.4% |
| Autoantibodies | 0.60 ± 0.08 | 65.0% ± 3.4% | 70.0% ± 6.8% | 60.0% ± 5.6% | 63.7% ± 2.9% | 66.9% ± 4.5% |

^{α}Four types of classifiers were trained for each classification task: “multi-omics” used all omic features for network inference, feature selection, and classifier training, while “Proteins”, “Metabolites”, and “Autoantibodies” solely used their respective components. The classifier was tested using various cutoffs of subnetwork sizes (i.e., total number of nodes or features) and performance metrics were reported based on the optimal (i.e., highest accuracy) subnetwork size. The number of study participants in the ACPA− RA, ACPA+ RA, controls, and total RA (ACPA− RA and ACPA+ RA combined) are 40, 40, 40, and 80, respectively.
^{β}AUC, area under the receiver operating characteristic curve.
^{γ}PPV, positive predictive value.
^{δ}NPV, negative predictive value. Instances where the denominator in the NPV formulas would be zero are excluded from the calculation.

TABLE 3
ACPA− vs. ACPA+ RA classification performance in two independent plasma metabolomic datasets.

| | | Classification | AUC^{β} | Accuracy | Sensitivity | Specificity | PPV | NPV |
|---|---|---|---|---|---|---|---|---|
| Dataset | Description | task^{α} | (Mean ± SD) | (Mean ± SD) | (Mean ± SD) | (Mean ± SD) | (Mean ± SD) | (Mean ± SD) |
| 1 | Mayo Clinic Early | ACPA− RA vs. | 0.70 ± 0.07 | 66.1% ± 7.3% | 66.7% ± 0.0% | 65.4% ± 14.6% | 67.2% ± 9.7% | 65.4% ± 5.8% |
| | Arthritis Cohort^{γ} | ACPA+ RA | | | | | | |
| 2 | Hur et al. (2021)^{δ} | ACPA− RA vs. | 0.72 ± 0.04 | 68.6% ± 4.0% | 71.4% ± 0.0% | 65.9 ± 8.0% | 68.0% ± 5.2% | 69.1% ± 2.8% |
| | | ACPA+ RA | | | | | | |

^{α}Classification performance was evaluated using a model whose optimal subnetwork size and random seed were determined through 5-fold cross-validation on the training data.
^{β}AUC, area under the receiver operating characteristic curve.
^{γ}Unpublished cohort including newly diagnosed (i.e., disease duration ≤6 months), DMARD treatment-naïve 9 ACPA− and 28 ACPA+ RA patients.
^{δ}Published cohort including 28 ACPA− and 103 ACPA+ RA patients (see reference 28).

### REFERENCES

1. Aletaha, D., & Smolen, J. S. (2018). JAMA. 320(13):1360-1372.
2. Guo, Q., Wang, Y., Xu, D., Nossent, J., Pavlos, N. J., & Xu, J. (2018). Bone Res. 6:15.
3. Smolen, J. S., et al. (2018). Nat Rev Dis Primers. 4:18001.
4. Aletaha, D., et al. (2010). Ann Rheum Dis. 69(9):1580-1588.
5. Rheumatoid Arthritis (2018) Nat Rev Dis Primers. 4:18002.
6. Firestein G. S. (2003). Nature. 423(6937):356-361
7. Li, K., Wang, M., Zhao, L., Liu, Y., & Zhang, X. (2022). EBioMedicine. 83:104233.
8. Aggarwal, R., Liao, K., Nair, R., Ringold, S., & Costenbader, K. H. (2009). Arthritis Rheum. 61(11):1472-83.
9. Sieghart, D., et al. (2018). Front Immunol. 9:876.
10. Pruijn, G. J., Wiik, A., & van Venrooij, W. J. (2010). Arthritis Res Ther. 12(1):203.
11. Weyand, C. M., & Goronzy, J. J. (2021). Nat Immunol. 22(1):10-18.
12. Combe, B., et al. (2017). Ann Rheum Dis. 76(6):948-959.
13. Myasoedova, E., Davis, J., Matteson, E. L., & Crowson, C. S. (2020). Ann Rheum Dis. 79(4):440-444.
14. Seegobin, S. D., et al. (2014). Arthritis Res Ther. 16(1):R13.
15. Boer, A. C., Boonen, A., & van der Helm van Mil, A. H. M. (2018). Arthritis Care Res (Hoboken). 70(7):987-996.
16. Padyukov, L., et al. (2011). Ann Rheum Dis. 70(2):259-65.
17. He, J., Chu, Y., Li, J., Meng, Q., et al. (2022). Sci Adv. 8(6):eabm1511.
18. Cunningham, K. Y., et al. (2023). Sci Rep. 13(1):5360.
19. Han, P., Hou, C., et al. (2022). Front Immunol. 13:884462.
20. Lay Jr, J. O., Liyanage, R., Borgmann, S., & Wilkins, C. L. (2006). TrAC Trends Anal Chem. 25(11):1046-1056.
21. Momeni, Z., Hassanzadeh, E., Saniee Abadeh, M., & Bellazzi, R. (2020). J Biomed Inform. 107:103466.
22. Hasin, Y., Seldin, M., & Lusis, A. (2017). Genome Biol. 18(1):83.
23. Jiang, Y., Zhong, S., He, S., Weng, J., Liu, L., Ye, Y., & Chen, H. (2023). Front Immunol. 14:1087925.
24. Cheng, Y., Chen, Y., Sun, X., Li, Y., Huang, C., Deng, H., & Li, Z. (2014). Inflammation. 37(5):1459-67.
25. Prevoo, M. L., van't Hof, M. A., Kuper, H. H., van Leeuwen, M. A., van de Putte, L. B., & van Riel, P. L. (1995). Arthritis Rheum. 38(1):44-8.
26. Inoue, E., Yamanaka, H., Hara, M., Tomatsu, T., & Kamatani, N. (2007). Ann Rheum Dis. 66(3):407-409.
27. Sedlacek, R., Mauch, S., Kolb, B., Schätzlein, C., Eibel, H., Peter, H. H., Schmitt, J., & Krawinkel, U. (1998). Immunobiology. 198(4):408-423.
28. Hur, B., Gupta, V. K., Huang, H., Wright, K. A., Warrington, K. J., Taneja, V., Davis, J. M., 3rd, & Sung, J. (2021). Arthritis Res Ther. 23(1):164.
29. Peng, Y. F., Wang, J. L., & Pan, G. G. (2017). Clin Chim Acta. 469:187-190.
30. Juping, D., Yuan, Y., Shiyong, C., Jun, L., Xiuxiu, Z., Haijian, Y., Jianfeng, S., & Bo, S. (2017). J Clin Lab Anal. 31(6):e22118.
31. Bhattacharya, S., et al. (2018). Sci Data. 5:180015.
32. Rump, L., Mattey, D. L., Kehoe, O., & Middleton, J. (2017). Cytokine. 97:133-140.
33. Floudas, A., Canavan, M., McGarry, T., Mullan, R., Nagpal, S., Veale, D. J., & Fearon, U. (2021). Cells. 10(3):647.
34. Jiao, X., Sherman, B. T., Huang, daW., Stephens, R., Baseler, M. W., Lane, H. C., & Lempicki, R. A. (2012). Bioinformatics. 28(13):1805-1806.
35. Johnson, A. A., & Stolzing, A. (2019). Aging Cell. 18(6):e13048.
36. Hornburg, D., Wu, S., Moqri, M., et al. (2023). Nat Metab. 5(9):1578-1594.
37. Lei, Q., Yang, J., Li, L., Zhao, N., Lu, C., Lu, A., & He, X. (2023). Front Immunol. 14:1190607.
38. McGrath, C. M., & Young, S. P. (2015). Curr Rheumatol Rep. 17(9):57.
39. Mackay I. R. (2010). Autoimmun Rev. 9(5):A251-A258.
40. Suurmond, J., & Diamond, B. (2015). Clin Invest. 125(6):2194-2202.
41. Yue, C., Gao, S., Li, S., Xing, Z., Qian, H., Hu, Y., Wang, W., & Hua, C. (2022). Front Immunol. 13:911919.
42. Zhao, W., Dong, Y., Wu, C., Ma, Y., Jin, Y., & Ji, Y. (2016). Exp Cell Res. 340(1):132-138.
43. Napier, B. A., Brubaker, S. W., Sweeney, T. E., Monette, P., Rothmeier, G. H., Gertsvolf, N. A., Puschnik, A., Carette, J. E., Khatri, P., & Monack, D. M. (2016). J Exp Med. 213(11):2365-2382.
44. Song, J. J., Hwang, I., Cho, K. H., Garcia, M. A., et al. (2011). J Clin Invest. 121(9):3517-3527.
45. Okroj, M., Heinegård, D., Holmdahl, R., & Blom, A. M. (2007). Ann Med. 39(7):517-530.
46. Holers, V. M., & Banda, N. K. (2018). Front Immunol. 9:1057.
47. Di Muzio, G., Perricone, C., et al. (2011). Int J Immunopathol Pharmacol. 24(2):357-366.
48. Trouw, L. A., Haisma, E. M., Levarht, E. W., van der Woude, D., loan-Facsinay, A., Daha, M. R., Huizinga, T. W., & Toes, R. E. (2009). Arthritis Rheum. 60(7):1923-1931.
49. Wu, C. Y., Yang, H. Y., & Lai, J. H. (2020). Int J Mol Sci. 21(11):4015.
50. Chen, W., Wang, Q., Zhou, B., Zhang, L., & Zhu, H. (2021). Front Pharmacol. 12:643520.
51. Choy, E., & Sattar, N. (2009). Ann Rheum Dis. 68(4):460-469.
52. García-Gómez, C., Nolla, J. M., Valverde, J., Gómez-Gerique, J. A., Castro, M. J., & Pintó, X. (2009). J Rheumatol. 36(7):1365-1370.
53. Yan, J., Yang, S., et al. (2023). Front Immunol. 14:1254753.
54. Robinson, G., Pineda-Torra, I., Ciurtin, C., & Jury, E. C. (2022). J Clin Invest. 132(2):e148552.
55. Sung, J., Wang, Y., Chandrasekaran, S., Witten, D. M., & Price, N. D. (2012). Biotechnol J. 7(8):946-957.
56. Candia, J., Daya, G. N., Tanaka, T., Ferrucci, L., & Walker, K. A. (2022). Sci Rep. 12(1):17147.
57. Zou, H. and Hastie, T., 2005. J R Stat Soc Series B Stat Methodol. 67(2):301-320.
58. Valdeolivas, A., Tichit, L., Navarro, C., Perrin, S., Odelin, G., Levy, N., Cau, P., Remy, E., & Baudot, A. (2019). Bioinformatics. 35(3):497-505.

1. A method of treating a subject afflicted with rheumatoid arthritis (RA), said method comprising the steps of diagnosing the subject with rheumatoid arthritis (RA), comprising:
(a) obtaining a blood sample from the subject; and
(b) determining the amount of a plurality of biomolecules comprising plasma proteins, plasma metabolites and plasma autoantibodies in the blood sample; wherein
(i) the amount of at least 25 plasma proteins from Table A, and/or at least 25 plasma metabolites from Table B, and/or at least 25 plasma autoantibodies from Table C, or a combination of at least 50 biomolecules from Tables A, B and C, is higher in the blood sample relative to an amount of the biomolecules in a control sample from a subject without RA and indicates the subject has seronegative RA (ACPA− RA); or
(ii) the amount of at least 25 plasma proteins from Table D, and/or at least 25 plasma metabolites from Table E, and/or at least 25 plasma autoantibodies from Table F, or a combination of at least 50 biomolecules from Tables D, E and F, is higher in the blood sample relative to an amount of the biomolecules in a control sample from a subject without RA indicates the subject has seropositive RA (ACPA+ RA); and
(c) administering at least one therapy to the subject.

2. (canceled)

3. (canceled)

4. The method according to claim 1, wherein the determining the amount of biomolecules in step (b) produces a multi-omic data set, wherein the multi-omic data set is processed through a trained machine learning model, and wherein the processed multi-omic data set provides a RA phenotype classification.

5. A method of diagnosing a subject with rheumatoid arthritis (RA), said method comprising the steps of preparing a RA phenotype classification network comprising:
(a) receiving multi-omic data from a blood sample from a subject, wherein the multi-omic data includes proteomic, metabolomic, and autoantibody profiles;
(b) processing the received multi-omic data through a trained machine learning model;
(c) classifying the subject's RA phenotype based on the processed multi-omic data; and
(d) outputting the classification result; and wherein:
(i) the classification is ACPA− RA when the amount of at least 25 plasma proteins from Table A, and/or at least 25 plasma metabolites from Table B, and/or at least 25 plasma autoantibodies from Table C, or a combination of at least 50 biomolecules from Tables A, B and C is detected; or
(ii) the classification is ACPA+ RA when the amount of at least 25 plasma proteins from Table D, and/or at least 25 plasma metabolites from Table E, and/or at least 25 plasma autoantibodies from Table F, or a combination of at least 50 biomolecules from Tables D, E and F is detected.

6. The method of claim 5, wherein the trained machine learning model is trained by
(a) selecting a cohort of subjects comprising subgroups of ACPA− RA patients, ACPA+ RA patients, and control individuals without RA;
(b) collecting blood samples from the selected cohort;
(c) performing deep multi-omic profiling on the collected blood samples, wherein the deep multi-omic profiling includes proteomic profiling, metabolomic profiling, and autoantibody profiling;
(d) analyzing the multi-omic data to identify biomolecular features associated with each RA subgroup through statistical analyses, set comparisons, and network inference techniques;
(e) constructing a global multi-omic network based on the identified biomolecular features and their associations with clinical attributes, including RA phenotypes;
(f) applying a network diffusion technique to prioritize features closely linked to the RA phenotypes;
thereby training the machine learning model using the prioritized features to distinguish between ACPA− RA subjects, ACPA+ RA subjects, and control subjects.

7. A method of treating a subject with rheumatoid arthritis (RA), said method comprising the steps of:
(I) diagnosing a subject with rheumatoid arthritis (RA), said method comprising the steps of preparing a RA phenotype classification network comprising:
(a) receiving multi-omic data from a blood sample from a subject, wherein the multi-omic data includes proteomic, metabolomic, and autoantibody profiles;
(b) processing the received multi-omic data through a trained machine learning model;
(c) classifying the subject's RA phenotype based on the processed multi-omic data; and
(d) outputting the classification result; and wherein:
(i) the classification is ACPA− RA when the amount of at least 25 plasma proteins from Table A, and/or at least 25 plasma metabolites from Table B, and/or at least 25 plasma autoantibodies from Table C, or a combination of at least 50 biomolecules from Tables A, B and C is detected; or
(ii) the classification is ACPA+ RA when the amount of at least 25 plasma proteins from Table D, and/or at least 25 plasma metabolites from Table E, and/or at least 25 plasma autoantibodies from Table F, or a combination of at least 50 biomolecules from Tables D, E and F is detected; and
(II) administering at least one therapy to the subject

8. The method according to claim 1, wherein the subject is a mammal.

9. The method of claim 8 wherein the mammal is a human.

10. The method of claim 1, wherein the determining the amount of plasma proteins comprises an aptamer-based assay technique.

11. The method of claim 10, wherein the determining the amount of plasma proteins comprises an aptamer-based assay that measures binding of target proteins to aptamers in relative fluorescent units (RFUs).

12. The method according to claim 1, wherein the determining the amount of plasma metabolites comprises a mass spectrometry technique.

13. The method of claim 12, wherein the determining the amount of plasma proteins comprises ultra-high-performance liquid chromatography-tandem mass spectrometry (UPLC-MS/MS).

14. The method of according to claim 1, wherein the determining the amount of plasma autoantibodies comprises a microarray-based assay technique.

15. The method according claim 1, wherein the therapy comprises administering one or more of a small molecule agent, immunotherapy, disease-modifying antirheumatic drugs (DMARDs), nonsteroidal anti-inflammatory drugs (NSAIDs), steroids, a biologic agent, and surgery.

16. The method of claim 15, wherein the therapy comprises ibuprofen and/or naproxen sodium.

17. The method of claim 15, wherein the therapy comprises a corticosteroid comprising one or more of cortisone, prednisolone, methylprednisolone, dexamethasone, betamethasone, and hydrocortisone.

18. The method of claim 15, wherein the therapy comprises at least one DMARD comprising methotrexate, leflunomide, hydroxychloroquine, and sulfasalazine.

19. The method of claim 15, wherein the therapy comprises at least one of abatacept, adalimumab, anakinra, certolizumab, etanercept, golimumab, infliximab, rituximab, sarilumab, tocilizumab, baricitinib, tofacitinib, and upadacitinib.

20. The method of claim 16, wherein the therapy comprises snynovectomy, tendon repair, joint fusion, and joint replacement.

21. The method according to claim 15, wherein 1, 2, 3, 4 or more therapies are administered.
